(19) United States
(12) Patent Application Publication
(10) Pub. No.: US 2008/0081990 A1
(43) Pub. Date: Apr. 3, 2008

(54) APPARATUS, SYSTEM, KIT AND METHOD FOR HEART MAPPING

(75) Inventors: Omer Berenfeld, Dewitt, NY (US); Jerome Kalifa, Syracuse, NY (US); Jose Jalife, Manlius, NY (US)

Correspondence Address: SCHMEISER, OLSEN & WATTS, 22 CENTURY HILL DRIVE, SUITE 302, LATHAM, NY, 12110, US

(73) Assignee: The Research Foundation of State University of New York, Albany, NY (US)

(21) Appl. No.: 11/863,599

(22) Filed: Sep. 28, 2007

Related U.S. Application Data
(60) Provisional application No. 60/827,227, filed on Sep. 28, 2006.

(51) Int. Cl. A61B 5/055; G06Q 50/00

(52) U.S. Cl. 600/420; 705/2

(57) ABSTRACT

The invention relates to a method of high-resolution mapping of a heart, including: providing a heart mapping apparatus; contacting at least a portion of an intact heart tissue with a voltage-sensitive fluoroscopic dye to generate at least a portion of dyed heart tissue; inserting a first end of the heart mapping apparatus into an intact heart; illuminating the portion of dyed heart tissue with a first range of wavelengths of electromagnetic radiation from the first end of the heart mapping apparatus; collecting a second range of wavelengths of electromagnetic radiation from the portion of dyed heart tissue; and transforming the second range of wavelengths of electromagnetic radiation to at least about 100 points of information, wherein said 100 points of information yields a map of at least one anatomical feature and at least one electrical potential.

### RELATED APPLICATION

The present invention is a non-provisional application which corresponds to U.S. Provisional Application No. 60/827,227 filed Sep. 28, 2006 and entitled “Endoscopic Mapping of Electrical Activity of the Heart”. The aforementioned application is incorporated herein by reference in its entirety.

### FUNDING STATEMENT

This invention was made with government support under contract identifier R01-HL60843 awarded by the National Heart and Blood Institute and non-government support under contract identifier 0230311N awarded by the American Heart Association. The government has certain rights in the invention.

### BACKGROUND OF THE INVENTION

1. Technical Field

The present invention relates to an apparatus, system, kit, and method for mapping the heart. More specifically, the present invention relates to mapping the anatomical features and electrical potential of heart tissue with a voltage-sensitive dye and mapping apparatus.

2. Related Art

Understanding the details of the dynamics and functions of the heart is a critical issue in prolonging human health and longevity. The heart is responsible for pumping oxygen rich blood to the entire body; therefore, if the heart is functioning sub-optimally, human health is often on the decline. Important variables in heart health include both the physical state of the heart tissue and also the electrical properties of the various cells of heart tissue. As cardiac muscle is myogenic muscle which can naturally contract and relax, understanding the voltage changes and electrical potential of heart cells affords clinicians, physicians, professionals, and researchers a better understanding of the electrical function and dynamic changes of a working heart. Visualization of both the electrical impulses in the heart with the underlying anatomy is useful in investigating cardiac arrhythmias that affect millions worldwide.

Current technology for mapping the heart provides a low space resolution data, little to no anatomical feature information, inoperability in tight or small spaces, inadequate electrical potential information, and inadequate information about an in situ heart dynamically functioning in an in vivo organism. Hence, there exists a need for an apparatus, method, system, and kit for high resolution mapping the anatomical features and electrical potential of the heart, operable under various conditions and applications, where the measurements, including images and maps, are instantaneously (real time feedback) available for screening, diagnostics, observation, manipulation, or other use.

### SUMMARY OF THE INVENTION

A first aspect of the present invention provides a heart mapping apparatus, comprising: an electromagnetic radiation source capable of exciting a fluoroscopic dye, said dye configured to emit at least an emission wavelength of electromagnetic radiation; a posable tubing, configured to associate with said electromagnetic radiation source, said posable tubing including: a delivery channel operable to deliver said excitation wavelength of electromagnetic radiation from said electromagnetic radiation source to a portion of heart tissue dyed with said fluoroscopic dye and an acquisition channel operable to acquire electromagnetic radiation having at least an emission wavelength defined by electromagnetic radiation, said emission wavelength emitted from said portion of heart tissue dyed with said fluoroscopic dye; an acquisition member, configured to receive the emission wavelength and transform the emission wavelength into a data stream having at least 100 points of information.

A second aspect of the present invention provides a method of high-resolution mapping of a heart, comprising: providing a heart mapping apparatus; contacting at least a portion of an intact heart tissue with a voltage-sensitive fluoroscopic dye to generate at least a portion of dyed heart tissue; inserting a first end of said heart mapping apparatus into an intact heart; illuminating said portion of dyed heart tissue with a first range of wavelengths of electromagnetic radiation from said first end of said heart mapping apparatus; collecting a second range of wavelengths of electromagnetic radiation from said portion of dyed heart tissue; and transforming said second range of wavelengths of electromagnetic radiation to at least about 100 points of information, wherein said 100 points of information yields a map of at least one anatomical feature and at least one electrical potential.

A third aspect of the present invention provides a system of high-resolution endocardial mapping, comprising: a predetermined amount of voltage-sensitive fluoroscopic dye, configured to absorb into at least a portion of heart tissue; a heart mapping apparatus, configured to cooperate with said dye to excite said dye such that said dye emits an emission wavelength range higher than the excitation wavelength range, said heart mapping apparatus further configured to transform said emission wavelength to computer usable data; and a computer system comprising an algorithm, said computer system including an algorithm, said computer system connected to said heart mapping apparatus and configured to received said computer usable data and display said data as a simultaneous anatomical features map and an electrical potential map of said portion of heart tissue.

A fourth aspect of the present invention provides a heart mapping kit, comprising: a predetermined quantity of voltage-sensitive fluorescing dye; an administration device for administering to a subject said predetermined quantity of voltage-sensitive fluorescing dye; a heart mapping apparatus, configured to enter an intact heart and take in-situ measurements, further configured to transform an electromagnetic radiation measurement into a data stream; and a computational tool, configured to accept said data stream from the heart mapping apparatus and allow a user to analyze, manipulate, and report a result from said data stream.

### BRIEF DESCRIPTION OF THE DRAWINGS

The present invention is described in detail below with reference to the drawings in which:

FIG. 1 depicts a flowchart of an example of an embodiment of the heart mapping apparatus of the present invention;

FIG. 2 depicts a flowchart of another example of an embodiment of the heart mapping apparatus of the present invention;

FIG. 3 depicts an example of an embodiment of the heart mapping apparatus of the present invention;

FIG. 4 depicts another example of an embodiment of the heart mapping apparatus of the present invention;

FIG. 5 depicts a front view of still yet another example of an embodiment of the heart mapping apparatus of the present invention;

FIG. 6 depicts a side view of further still another example of an embodiment of the heart mapping apparatus of the present invention;

FIG. 7 depicts a cut-away perspective side view of yet still another example of an embodiment of the heart mapping apparatus of the present invention;

FIG. 8 depicts an in situ cut-away side view of an example of the heart mapping apparatus of the present invention in relation to a surface portion of tissue;

FIG. 9 depicts a flowchart of an example of an embodiment of the method of high-resolution heart mapping of the present invention;

FIG. 10 depicts a flowchart of another example of an embodiment of the method of high-resolution heart mapping of the present invention;

FIG. 11 depicts a flowchart of still another example of an embodiment of the method of high-resolution heart mapping of the present invention;

FIG. 12 depicts a flowchart of still yet another example of an embodiment of the method of high-resolution heart mapping of the present invention;

FIG. 13 depicts a flowchart of further still another example of an embodiment of the method of high-resolution heart mapping of the present invention;

FIG. 14 depicts an in situ cut-away side view of a heart with an example of the heart mapping apparatus of the present invention at least partially inserted therein;

FIG. 15 depicts an in situ cut-away side view of a heart with an example of the heart mapping apparatus of the present invention at least partially inserted therein;

FIG. 16A depicts an image taken from the inside of the left atrium of a subject heart;

FIG. 16B depicts an image taken from the inside of the left atrium of a subject heart;

FIG. 16C depicts an image taken from the inside of the left atrium of a subject heart;

FIG. 16D depicts an illustration of a surgical opening and three images taken from the inside of the left atrium of a subject heart;

FIG. 17A depicts a fluorescence image of the lower PLA-appendage junction and consecutively obtained frames;

FIG. 17B depicts single pixel recordings at various locations of the fluorescence image frame 19 of FIG. 17A;

FIG. 18A depicts an endoscopic view of the interior of the left atrium;

FIG. 18B depicts a clockwise micro-reentrant activity in a snapshot from a phase movie;

FIG. 18C depicts a single pixel recording in the left atrium;

FIG. 18D depicts the micro-reentrant activity after transition into spatio-temporally organized waves traveling in the septal direction (arrows indicate direction);

FIG. 19A depicts a sinus wave propagation on the pectinate muscles;

FIG. 19B depicts a sinus wave propagation on the pectinate muscles;

FIG. 19C depicts a sinus wave propagation on the pectinate muscles;

FIG. 19D illustrates the orientation and focus of the apparatus for FIG. 19A, FIG. 19B, and FIG. 19C;

FIG. 20 depicts the visualization of the RF energy delivery effect on sinus rhythm (SR) impulse propagation at the posterior left atrium;

FIG. 21A depicts two signal to noise (SNR) maps for atrial fibrillation video recorded;

FIG. 21B depicts signal to noise (SNR) histograms during AF for and filtered data;

FIG. 21C provides a table listing the signal to noise (SNR) averages calculated from the Full-width half height (FWHH) range;

FIG. 22 depicts the structural formula of some of the examples of the voltage-sensitive dyes utilized with one or more aspects of the present invention;

FIG. 23 depicts an example of an embodiment of a system of high-resolution heart mapping of the present invention;

FIG. 24 depicts an example of a computer system of an example of a system of high-resolution heart mapping of the present invention; and

FIG. 25 depicts an example of an embodiment of a heart mapping kit of the present invention.

### DETAILED DESCRIPTION OF THE INVENTION

The present invention provides various embodiments used in the heart mapping field, as well as for the screening of heart conditions, including diseases and illnesses. Such heart conditions include, for example, atrial fibrillation (AF is the most common sustained arrhythmia seen in clinical practice), sinus rhythm, arrhythmia, valvular heart disease, diseased or otherwise ineffective heart tissue, etc. . . . Although an apparatus, system, kit and method for mapping heart tissue will be discussed and disclosed in detail inter alia, it should be understood by those skilled in the art that the applications referenced here and the various embodiments of the present invention may be used throughout the body for mapping of the electrical potential of various portions of tissue.

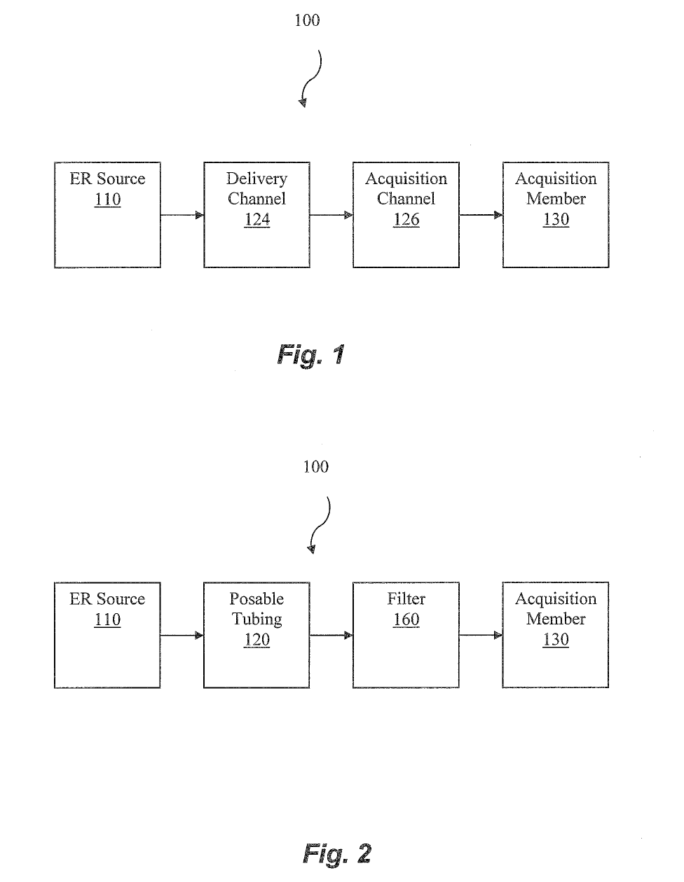

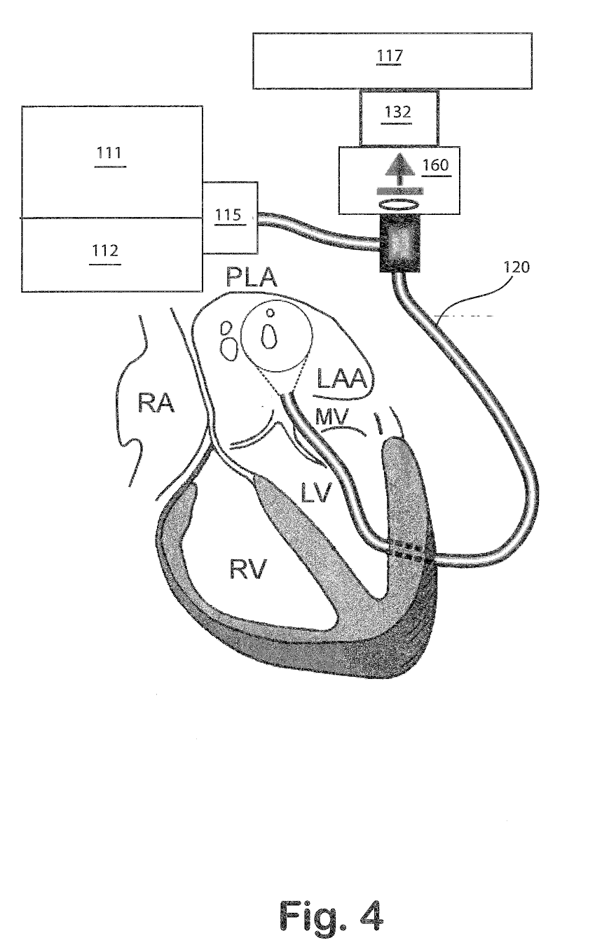

The heart mapping apparatus 100 of the present invention in its various embodiments and examples may be depicted, for example, in FIG. 1 through FIG. 8. The heart mapping apparatus 100 may comprise: an electromagnetic radiation source 110, a posable tubing 120, and an acquisition member 130, as depicted in FIG. 1. FIG. 4 depicts the heart mapping apparatus showing an experimental set-up, as the apparatus 100 is inserted into the left atrium through a minimal left ventricular opening, and across the mitrial valve. The various elements and features of the heart mapping apparatus are disclosed and described in the paragraphs that follow.

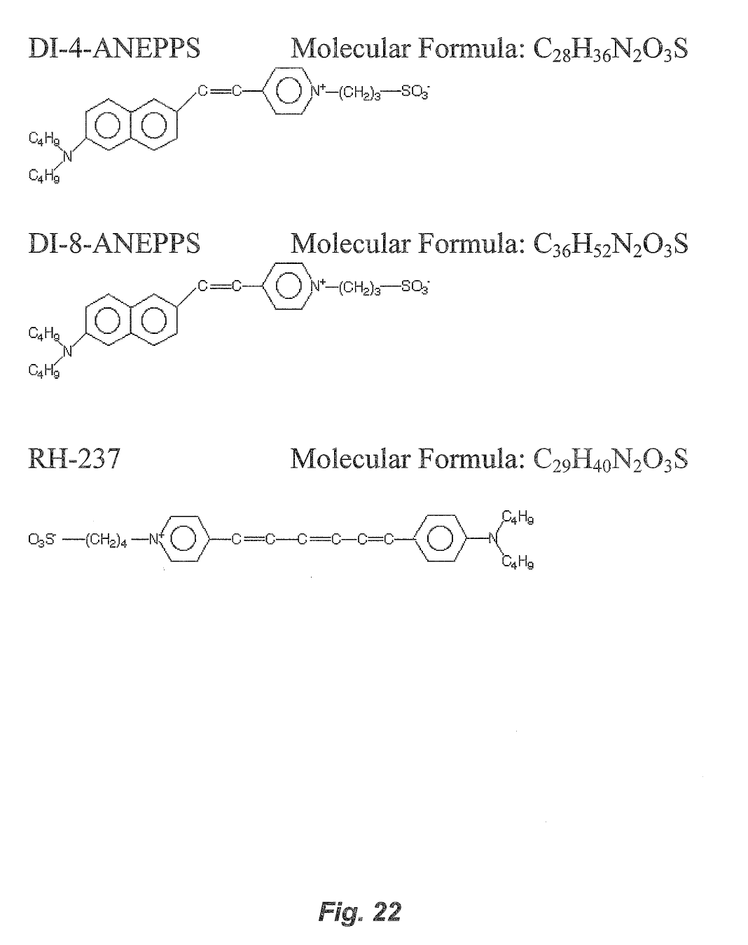

The heart mapping apparatus 100 of the present invention may comprise an electromagnetic radiation (ER) source 110, where the ER source 110 may be capable of exciting a fluoroscopic dye 190 as, for example, one of the dyes listed in FIG. 22, or the dye staining the heart tissue surface 70 in FIG. 8. The ER source 110 may be a visible light source or alternatively an infrared light source. The ER source 110 may comprise, for example, one or more light bulbs in which the light source produces and directs visible light. As another example, the ER source 110 may comprise a laser 111, where the laser 111 is an optical device that produces an intense monochromatic beam of coherent light. For example, the laser 111 may be a 532 nm excitation Laser used as an ER source 110 for illumination. The laser 111 may operate to produce light at a given wavelength of electromagnetic light. For example, the ER source 110 laser 111 may produce and direct laser light which may transmitted and utilized by one or more elements of the heart mapping apparatus 100. Also, the ER source 110 may be a combined laser 111 and visible light source 112 with, for example, a toggle switch 115 such that one or the other type of electromagnetic radiation may be produced and directed into another portion of the apparatus. The heart mapping apparatus may further comprise a display 117, which may work, for example, in conjunction with a computer system 330 (as described and depicted infra), a video recorder, a television screen, a computer monitor, et cetera.

The ER source 110 may be configured to excite a dye, where the dye exhibits certain characteristic properties of for example, absorption max, excitation max, solubility limits, and/or intensity of excitation. The dye may be a fluoroscopic dye, or a voltage-sensitive fluoroscopic dye 190. One or more types of dye that may be used with the apparatus will be discussed and disclosed infra. The ER source 110 may be capable of exciting a voltage-sensitive fluoroscopic dye 190, such that voltage-sensitive fluoroscopic dye 190 may emit at least an emission wavelength 50 of electromagnetic radiation.

The voltage-sensitive fluoroscopic dye 190 may be employed as a contrast agent. Voltage-sensitive fluoroscopic dye refers to a probe that is readily affected by an electric potential. The dye is a chemical substance which has an affinity for a substrate, such as heart tissue and emits fluorescence upon exposure to an electromagnetic radiation having a specific wavelength. Thus, dye may be applied in vivo and absorb into a portion of tissue and once the portion of dyed tissue is exposed to an excitation wavelength or electromagnetic radiation source, the fluorescence at a range of electromagnetic radiation is emitted from the dye, and the patterning of the dye in the tissue is detected. While fluorescent dyes emit various wavelengths of electromagnetic radiation resulting in various colors, living tissue only emits light of about 400 to 600 nm; therefore, transmission through living tissue at these wavelengths is very low so detection is near impossible.

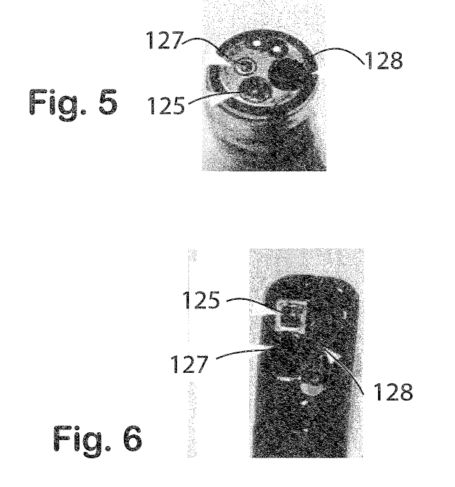

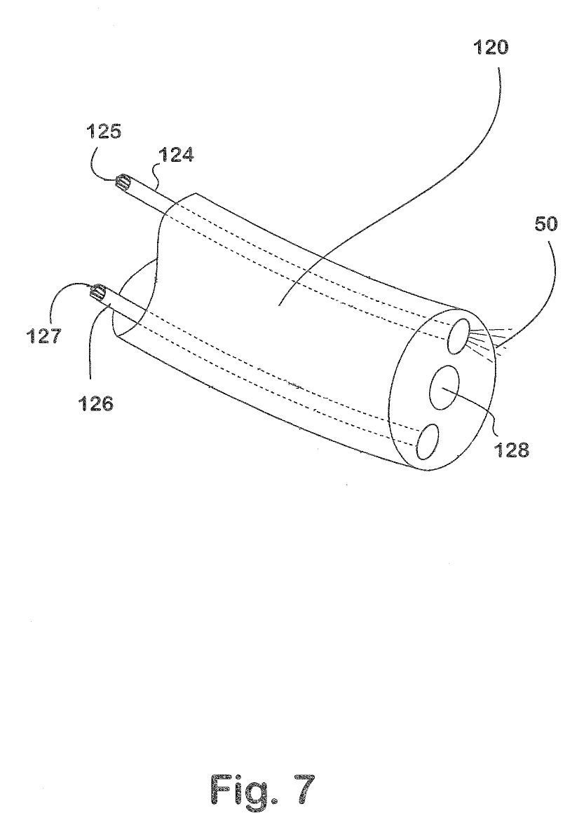

The heart mapping apparatus 100 further comprises a posable tubing 120. The posable tubing 120 may be, for example, an endoscope, a catheter 122, or an endoscopic catheter. For example, the posable tubing may be either a direct view endoscope or side-view endoscope. The posable tubing 120 may be composed one or more various materials known to those in the art, including, for example: metal, metal alloys, plastic, polymer, vinyl, a fluoropolymer such as polytetrafluoroethylene (PTFE) (e.g. material sold under the trade name Teflon®), or the like. The posable tubing 120 may include one integral tubing, or a plurality of parts that cooperate to form a posable tubing 120. The posable tubing 120 may be of a given length, for example, a meter, two meters, or meters in length. The posable tubing 120 may be of a long, small configuration, such that the posable tubing 120 is readily insertable into small, tight places, including in vivo application into, for example, the circulatory system. The tubing is flexible such that it may bend when inserted into a body's passageway and has a tip which may be positioned in the coronary sinus. The tip may further be posable, and may be equipped with wide fisheye lenses to provide increased visibility of the portion of heart tissue. In substantially cylindrical formation, the cross section of the tubing may be, for example, a millimeter in diameter up a centimeter or more in diameter. The posable tubing 120 may be configured to associate with said electromagnetic radiation source 110. The posable tubing 120 may include, for example, a delivery channel 124 and an acquisition channel 126, as shown in FIG. 1, FIG. 5, FIG. 6, FIG. 7 and FIG. 8.

The delivery channel 124 of the posable tubing 120 may be operable to deliver an excitation wavelength 50 of electromagnetic radiation from said electromagnetic radiation source 110 to a portion of heart tissue 70 dyed with said fluoroscopic dye 190. Further, the delivery channel 124 may be a single fiber optic cable 125 or a plurality of cables bundled together. In such a manner, the excitation wavelength 50 made by the ER source 110 may in turn be transmitted from the delivery channel 124 to a portion of heart tissue 70.

The posable tubing 120 may further include an acquisition channel 126. The acquisition channel 126 may be operable to acquire electromagnetic radiation having at least an emission wavelength 60 defined by electromagnetic radiation, where the emission wavelength 60 may be emitted from said portion of heart tissue 70 dyed with said fluoroscopic dye 190. That is, the acquisition channel 126 may sense the emission wavelength from the heart tissue 70 and transmit the emission wavelength 60 back through the posable tubing 120. Further, the acquisition channel 126 may be a single fiber optic cable 127 or a plurality of cables bundled together.

The posable tubing 120 may include at least these two channels (delivery channel 124 and acquisition channel 126), but may include more, for example, a working channel 128 which may be configured to pose the posable tubing 120. That is, the working channel 128 may comprise robotics, mechanical elements, electromechanical elements, hydraulic elements, and the like known to those in the art which may commonly be utilized with endoscopic catheters such that the catheters may be negotiated through tight spaces and great precision and accuracy.

The heart mapping apparatus 100 may further comprise an acquisition member 130. The acquisition member 130 may be configured to receive electromagnetic radiation at the emission wavelength 60 and transform the emission wavelength 60 into a data stream 80 having at least 100 points of information 82. The acquisition member may comprise a charge coupled device (CCD) or complementary metal-oxide-semiconductor (CMOS) camera or photodiodes array 132. The camera or array 132 may, for example, image the potentiometric dye fluorescence at a given resolution. The resolution may be, for example, 80×80 pixels at a rate of, for example, 200-800 frames per second.

A heart mapping apparatus 100 may have an endoscope with a combination of optical properties including sufficient signal to noise ratio (SNR) and spatiotemporal resolving power to allow quantification of wave propagation. For example, by using an endoscope with greater than 11% transmittance in the relevant ranges, it is possible to achieve a SNR greater than 25. This SNR is less than 10% smaller than the SNR of a direct mapping system using the same light source and camera. One of the possible explanations that the endoscope's SNR is comparable to the direct mapping system despite its marked reduced light transmittance is the significantly smaller distance (˜1 cm) between the endoscopic objective lens and the tissue surface.

For better mechanical control of the posable tubing, one may use, for example, endoscopes which may be posable, flexible, steerable, and/or locking ability. That is, locking ability may contribute to better stabilization of the tubing of the apparatus. The endoscope may, for example, be introduced into the left atrium via a minimal incision in the left ventricular free wall. Alternatively, the endoscope may be introduced via a caval route (of, relating to, or characteristic of the vena cava) and transeptal puncture (passing or performed through a septum) to image the LA. The presence of a working channel for introduction of recording and ablation catheters (to facilitate the removal of abnormal growths or substances) helped maximize the applicability of the system used.

The heart mapping apparatus 100 may further comprise a filter 160. The filter 160 may be located either in the acquisition channel 126 or near an end of the acquisition channel 126 such that the filter 160 may reduce or eliminate from the acquisition channel one or more types of electromagnetic radiation. This may in turn reduce scattering, and improve the signal to noise ratio of the heart mapping apparatus 100 in use. An example of a filter 160 may include a quasi-monochromatic filter 161. The quasi-monochromatic filter 161 may be placed at the end of the posable tubing 120 such that electromagnetic radiation collected with the acquisition channel 126 may be filtered prior to transmission of the ER radiation to the acquisition member 130, or CCD or CMOS camera or photodiodes array 132. With a quasi-monochromatic filter 161 in place, the excitation wavelength 50 and emission wavelength 60 electromagnetic radiation which may be together irradiated into the acquisition channel 126 may be filtered such that only the emission wavelength 60 is transmitted to the acquisition member 130 for depiction. Also, as the fluorescing dye may absorb light at a lower wavelength or range of wavelengths than it emits light in its excited state, a monochromatic filter may provide a means of increasing the clarity, precision, and accuracy of the image by reducing and interference which may come from bodily fluid, tissue, or bone absorption of the electromagnetic radiation at the excitation wavelength.

The heart mapping apparatus 100 in operation may be aimed at one or more successive locations throughout various portions of the heart, including the atrium and ventricle walls. When the heart mapping apparatus 100 may be used in combination with a voltage-sensitive dye, the potentiometric aspect of the heart cells may be measured to yield or otherwise obtain high resolution still images or live movies of, for example, electrical wave propagation through the heart tissue cells, as well as detailed endocardial anatomical features, in the presence and the absence of various heart conditions, including, for example, atrial stretch.

The posable tubing 120 may be connected at a second end of the tubing such that the delivery channel 124 is connected, in near proximity to or otherwise associated with an ER source 110. Also, the posable tubing 120 may be connected at a second of the tubing such that the acquisition channel may be connected to, in near proximity with, or otherwise associated to a fast CCD or CMOS camera or photodiodes array 132, while the orientation of the first end of the posable tubing 120 containing the acquisition channel 126 may be in a direction pointing substantially toward the endocardial surface of the heart. FIG. 5 and FIG. 6 depict a front plan view of the posable tubing in a direct-view configuration and side-view configuration, respectively, where the direct-view and side-view tips are clearly labeled to illustrate a configuration of the delivery channel 124, acquisition channel 126 and working channel 128. The posable tubing 120 may be equipped with the requisite lenses and sensors in order to properly position, image, and perform various other functions.

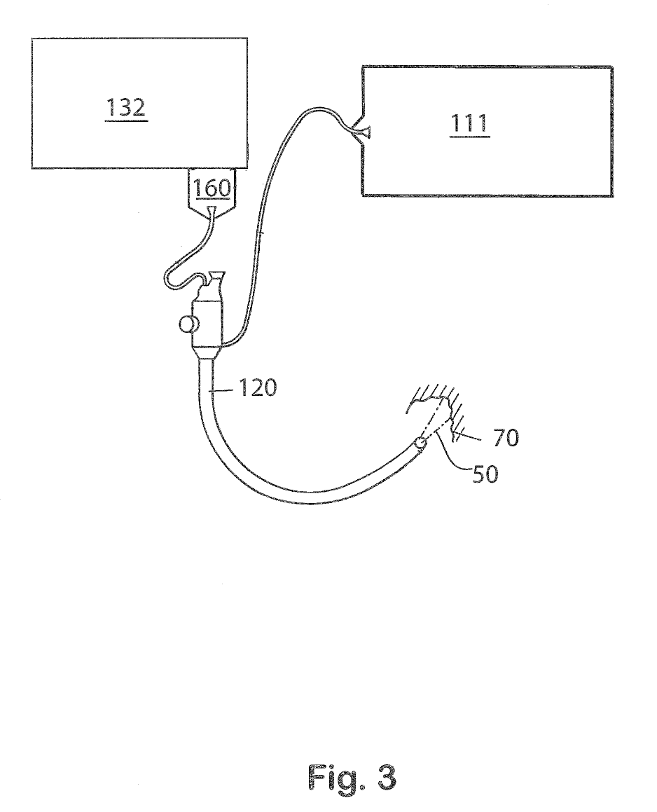

Also, as previously mentioned, quasi-monochromatic filters may be utilized in order to control the light frequency in both channels, as depicted in FIG. 2, FIG. 3, and FIG. 7. Also, though the delivery channel 124 passes light with a higher energy than the acquisition channel 126, both electromagnetic radiation channels may likewise be filtered with a quasi-monochromatic filter in order to reduce or eliminate the presence of electromagnetic radiation or light at undesirable wavelengths. The delivered light excites a fluoroscopic dye 190 in the heart tissue 70 and the emitted light is captured by the acquisition channel 60, free of interference with the excitation light as the channel is filtered.

As the intensity of the emitted light in the acquisition band-pass is proportional to the cells' transmembrane potential (electrical potential), the CCD or CMOS camera or photodiodes array 132 captures the electrical activity on the internal surface of the heart. The apparatus is innovative, fully operational, effective, and efficient as electrical activity or electrical potential of the internal surface of the heart may be captured in a video stream of data, ready for immediate display and analysis. The heart mapping apparatus 100 may map simultaneously, and with high resolution, the electrical activity and anatomical features of the internal surfaces of the intact heart, which may give a direct measure of the transmembrane potential of the cardiac cells, thereby allowing for more rigorous and relevant study of impulse propagation and dynamics of the heart during, for example, arrhythmia, sinus rhythm, or heart murmur.

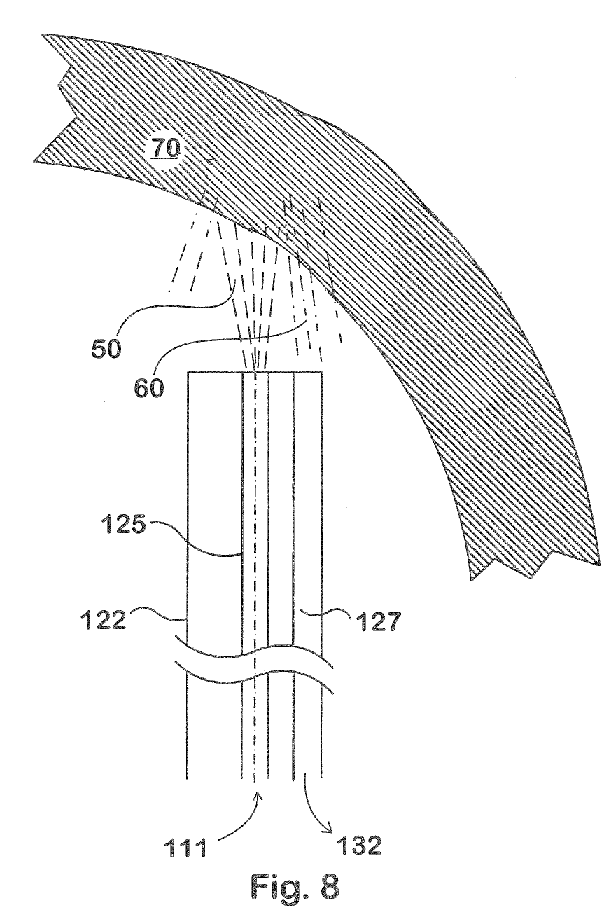

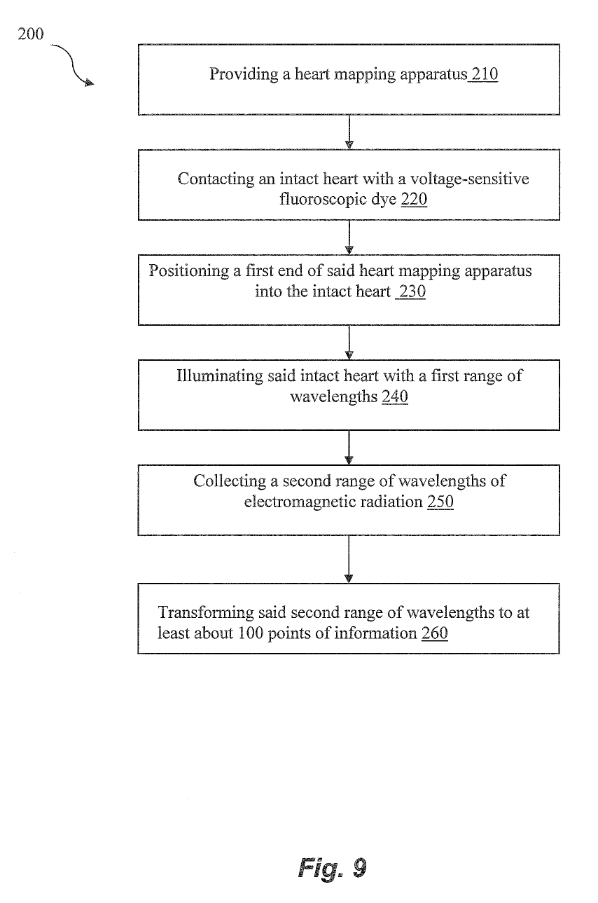

Another embodiment of the present invention includes a method for high-resolution mapping of a heart 200. Various examples of the embodiment may be depicted in FIG. 9 through FIG. 13. The method for high-resolution mapping of a heart 200 as depicted in FIG. 9 may comprise: providing a heart mapping apparatus 210; contacting at least a portion of intact heart tissue with a voltage-sensitive fluoroscopic dye to generate at least a portion of dyed heart tissue 220; inserting a first end of said heart mapping apparatus into an intact heart 230; illuminating said portion of intact heart tissue with a first range of wavelengths of electromagnetic radiation from said first end of said heart mapping apparatus 240; collecting a second range of wavelengths of electromagnetic radiation from said portion of dyed heart tissue with said first end of said heart mapping apparatus 250; and transforming said second range of wavelengths of electromagnetic radiation to at least about 100 points of recorded data from said second wavelength of electromagnetic radiation into a map of at least one anatomical feature and at least one electrical potential 260.

The method of high-resolution mapping of a heart 200 comprises the step of providing a heart mapping apparatus 110. The heart mapping apparatus 100 may include an ER source 110, a posable tubing 120 including a delivery channel 124 and an acquisition channel 126, and an acquisition member 130. The heart mapping apparatus 100 discussed supra, may be used in the method of high-resolution mapping of a heart 200.

The method of high-resolution mapping of a heart 200 also comprises the step of contacting at least a portion of intact heart tissue with a voltage-sensitive fluoroscopic dye 190 to generate at least a portion of dyed heart tissue 220.

Contacting, as referenced herein, includes physical contact of the voltage-sensitive fluoroscopic dye 190 to the surface of the heart tissue 70, which may include the endocardium and the epicardium. The voltage-sensitive fluoroscopic dye 190 may be distributed in close proximity to the heart wall by catheter dispersion, or by intravenous or injection via syringe or intravenous line. Contacting may also include, for example, direct injection of the dye into the heart tissue. Either close proximity distribution or direct contact/injection may be accomplished, for example, with an endoscopic catheter. The voltage-sensitive fluoroscopic dye 190 may be also consumed, imbibed, inhaled, or transdermally diffused into the subject and then transported by the circulatory system to the inner chambers of the heart and/or heart tissue. The voltage-sensitive dye may be contacted to the portion of heart tissue in a metered concentration, as photostability, solubuility, toxicity, and emission intensity may delineate. For example, a concentration of dye may be 10.4 μM dye dissolved in DMSO (dimethyl sulfoxide) and ringer solutions, and thereafter administered by contacting to a portion of heart tissue.

Intact heart tissue, as referenced herein, includes heart tissue which has not been anatomically modified or structurally compromised. Intact heart tissue may refer to a portion of heart tissue, a whole heart, a whole heart with portions of associated veins and arteries, and/or an in vivo heart which is fully operational and functioning.

The voltage-sensitive fluoroscopic dye 190, as referenced, herein, may refer to one or more of the group of dyes which absorb and excite from electromagnetic radiation at one wavelength and emit electromagnetic wavelength a higher wavelength than the excitation wavelength. Further, the voltage-sensitive dye may not excite at a measurable intensity when in solution with blood, lymph, and/or other bodily fluids, but may become excited at high intensity when the dye molecules are absorbed into the membrane of the heart tissue cells. The voltage-sensitive dye detects membrane potential in heart tissue cells, and areas with a greater membrane potential is exhibit a greater intensity of electromagnetic radiation which is emitted form the molecules of dye in the heart tissue.

The voltage-sensitive dye may be, for example, any of those with used or applicable in biological procedures. Classes of voltage-sensitive dyes which may be utilized in the present invention include, for example, styryl dyes, oxonol dyes, merocyanine-oxazolone dyes, and merocyanine-rhodanine dyes. As research and development in the area of fluorescing dyes and voltage-sensitive dyes is ongoing, various additional and alternative dyes may be applicable and utilizable in the present invention. Specifically, styryl dyes may include, for example, di-4-ANEPPS, di-8-ANEPPS, and RH237 where the structures and molecular formulas of the dyes are depicted in FIG. 22.

The ANEP (AminoNaphthylEthenylPyridinium) dyes are sensitive, exhibit relatively low toxicity, and are among the probes with the fastest response time. Di-4-ANEPPS (Molecular Formula: C28H36N2O3S) and di-8-ANEPPS (Molecular Formula: C36H52N2O3S) exhibit fairly uniform 10% per 100 mV changes in fluorescence intensity in a variety of tissue, cell and model membrane systems. Similar to the ANEP dyes, the RH dyes, including RH-237 (Molecular Formula: C29H40N2O3S) exhibit varying degrees of fluorescence excitation and emission spectral shifts in response to membrane potential changes. Their absorption and fluorescence spectra are also strongly dependent on the environment.

Di-4-ANEPPS, di-8-ANEPPS, and RH237 dyes as well as other styryl dyes typically absorb green light (at or around 500 nm), which excites the dyes and causes them to fluoresce at emission wavelengths typically in the red (600-650 nm) to the near infrared threshold (750 nm). Though many of the dyes currently absorb green light and emit red light, a dye with a characteristic near-IR, infrared, or far infrared fluorescence emission (when bound to membranes) may be beneficial in detection of emission electromagnetic radiation. That is, tissue or blood absorb more in the 400-600 nm range therefore light emitting at or around these ranges may be difficult to detect. However, if the emission wavelength was at a certain value in the range of 600-1800 nm, for example 1600 nm, the emission would exhibit better transmission through tissue and blood and exhibit a much greater detection by one or more acquisition members 130, including, for example, a fast CCD camera.

The portion of dyed heart tissue may comprise a small or large area of the heart, and may include, for example: a section of heart wall surface area, a given volume of heart tissue (dye absorption to a degree of thickness); an entire ventricle, an entire atria, an entire side of the heart, the entire heart, and combinations thereof. Also, there may be one or more portions of heart tissue that are dyed successively, simultaneously, or to differing degrees of concentration.

The method of high-resolution mapping of a heart 200 further comprises inserting a first end of said heart mapping apparatus into an intact heart 230. The inserting step may further comprise, for example, in vivo inserting of said first end of said heart mapping apparatus 100 into an entry point of a subject and following the circulatory system to the heart, said entry point selected from the group consisting of: a femoral artery, a jugular vein, and a brachial artery. The inserting step may comprise, as another example, inserting into the pulmonary artery, pulmonary vein, superior vena cava, inferior vena cava, of the wall of the right atrium, right ventricle, left atrium, or left ventricle a first end of the heart mapping apparatus 100.

The method of high-resolution mapping of a heart 200 next comprises the step of illuminating said portion of dyed heart tissue with a first range of wavelengths of electromagnetic radiation from said first end of said heart mapping apparatus 240. The first range of wavelengths of electromagnetic radiation may further comprise an excitation wavelength 50. The illuminating step may be done by operating the heart mapping apparatus 100 such that the ER source 110 generates an electromagnetic radiation of a particular wavelength or range of wavelengths and transmits the first range of wavelengths through the delivery channel 124 of the posable tubing 120, thereby distributing the electromagnetic radiation into a portion of the heart illuminating the surface of at least a portion of the heart tissue 70. For example, the illuminating step may further comprise illuminating the intact heart with said first range of wavelengths is about 500 to about 1800 nm.

The method of high-resolution mapping of a heart 200 further comprises the step of collecting a second range of wavelengths of electromagnetic radiation from said portion of dyed heart tissue with said first end of said heart mapping apparatus 250. The step of collecting 250 may further comprise transmitting by fiber optic bundle channel 252 the second range of wavelengths to a second end of the heart mapping apparatus 100. This may be done with, for example, the acquisition channel 126 of the posable tubing 120 of the heart mapping apparatus 100. As the second range of wavelengths is collected, it may be filtered in order to separate the excitation wavelength 50 from the emission wavelength 60, both of which may comprise the second range of wavelengths of electromagnetic spectrum. An example of a filter includes a quasi-monochromatic filter, which may remove excitation wavelengths and reflectance from other structures in the body. Once the second group of electromagnetic radiation is collected and filtered, a filtered range of wavelengths may be from the range of at least about 600 nm to about 1800 nm.

The method of high-resolution mapping of a heart 200 further comprises the step of transforming said second range of wavelengths of electromagnetic radiation to at least about 100 points of recorded data from said second wavelength of electromagnetic radiation into a map of at least one anatomical feature and at least one electrical potential 260. The transforming step may further comprise, for example, transforming by a CCD or CMOS camera or photodiodes array 132 the second range of wavelengths into a usable data. That is, the CCD or CMOS camera or photodiodes array 132 may transform either the second range of wavelengths without filtering or with filtering to yield the emission wavelengths range of electromagnetic radiation.

The at least about 100 points of recorded data may include references to pixels or points in a graphic image. The color depth of each point or pixel may be 8 bpp (bits per pixel), 16 bpp, 24 bpp, or 48 bpp depending on desired color depth. Or, reference to points may refer to the variable intensity of light that is reflected and captured from the surface of the heart tissue into the heart mapping apparatus 100. Further, the range of points of information yielded with the method of high-resolution mapping of a heart 200 may be from about 100 points to about 20,000 points. This is a vast improvement over previous technology, which only afforded up to 64 channels at optimum resolution, where a channel is not as clear as the pixels or points of information of the present invention. Reference to points may refer to pixel resolution. With a given apparatus, method, or system, the resolution may exceed 100 points of information, and may be, for example, 100, 1,000, 10,000 or 20,000 pixels. Also, it should be noted that the pixels are in extremely close proximity to one another, while other mapping systems, including those utilizing electrodes, place the electrodes as far apart as 1 cm, thereby reducing the accuracy and precision of electrical potential maps and anatomical feature maps. The apparatus disclosed herein may typically obtain measurements with a resolution at or exceeding 10,000 points of information or pixels.

The map of at least one anatomical feature and at least one electrical potential refers to the information gained about the heart tissue. Specifically, this may include locating anatomical features of the heart including bundles of heart tissue, locating areas of atrial fibrillation, locating veins and arteries, locating weak points of heart tissue, locating valve deficiencies, et cetera. Further, by taking the image or map from inside of the heart, it becomes easier to reconcile cross sectional movement for in vivo analysis (as the heart moves) by lining up the cross section of the anatomical features to understand how the heart tissue displaces during dynamic movement. The anatomical feature map may depict blood vessels, abnormal tissue configuration, tissue bundles, or non-unique homogenous heart tissue.

A map of the electrical potential of a portion of heart tissue will provide a detailed analysis and depiction of the places where a greater electrical potential exists. That is, heart conditions including atrial fibrillation and other arrhythmias may be diagnosed based on abnormal electrical wave propagation through heart tissue. As such, it may be possible to study and learn more about the electrical impulses in the heart, where the vast majority of the load originates from, how it is dissipated, and the dynamics of electrical measurements of membranes and understanding membrane potential of the heart in a dynamic movement.

By having a simultaneous depiction or map of the anatomical features and electrical potential of heart tissue, it is possible to garnish a better understanding of how anatomical features may be indicative of electrical potential differentiations of the heart, or vice versa. Further, the simultaneous map may be in a variety of different orientations with respect to the heart mapping apparatus measurement. That is, the simultaneous map may be a cross sectional view of the anatomical features of the heart and electrical potential of the heart tissue. Or, the simultaneous map may be a surface view of the interior of the heart, penetrating only a few cell layers deep. Or, the simultaneous map may be a three dimensional representation of the subject heart, such that the spatiotemporal and electrical potential are simultaneously depicted by, for example, computer modeling so illustrate how a user's heart functions both physically and electrically when in dynamic motion and operation.

Additionally, as the method of high-resolution mapping of a heart 200 may be performed in vivo, it should also be noted that as the method may be performed in a minimally invasive procedure, the subject may come in for periodic testing without much time, cost, pain, or recovery time. Also, the apparatus and method are such that the heart may be at peak pressure and readings acquired and images mapped from the method are still optimal. As is disclosed and discussed in the Examples section, the method of high resolution heart mapping 200 utilizing the heart mapping apparatus 100 may be done at real heart operating pressure and temperature conditions.

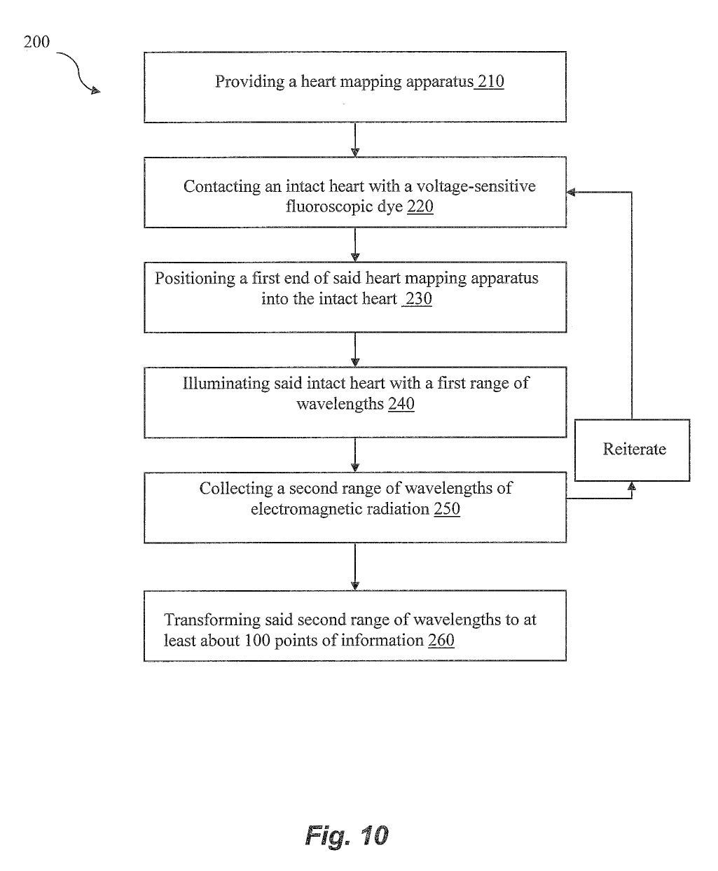

It should also be mentioned with reference to FIG. 10 through 13, one or more steps of the method of high-resolution mapping of a heart may be reiterated for any number of predetermined or otherwise calculated iterations such that a desired result is reached. That is, a subject may be under clinical advisement to undergo heart screening by the method of high-resolution heart mapping, for example, in a pre-determined time frame of a year in order to screen for potential heart conditions or diseases, while an individual may have to have calculated iterations, for example, if they are currently undergoing treatment for a condition and clinicians, professionals, or physicians cite a need to re-calculate or change the time frame by which an individual will have to screen or diagnose new, ongoing, or worsened conditions.

The reiterations of one or more steps may be indicative of the type of application in which the method is utilized. For example, with reference to FIG. 10, after the collecting step 250, the method may reiterate back to the dye-contacting step 220. That is, depending on the dye used, the solubility limit or wash out time may be such that long term data dynamic heart measurement may need to have additional dye samples delivered to one or more sites to be mapped. This may be important for applications including, for example, observation of drug efficacy over time. Therefore, after reiterating the contacting step, the remainder of the method is followed through to completion, wherein the inserting step may be inserting the first end of the catheter into another heart chamber, or repositioning the posable tubing in order to acquire one or more different measurements.

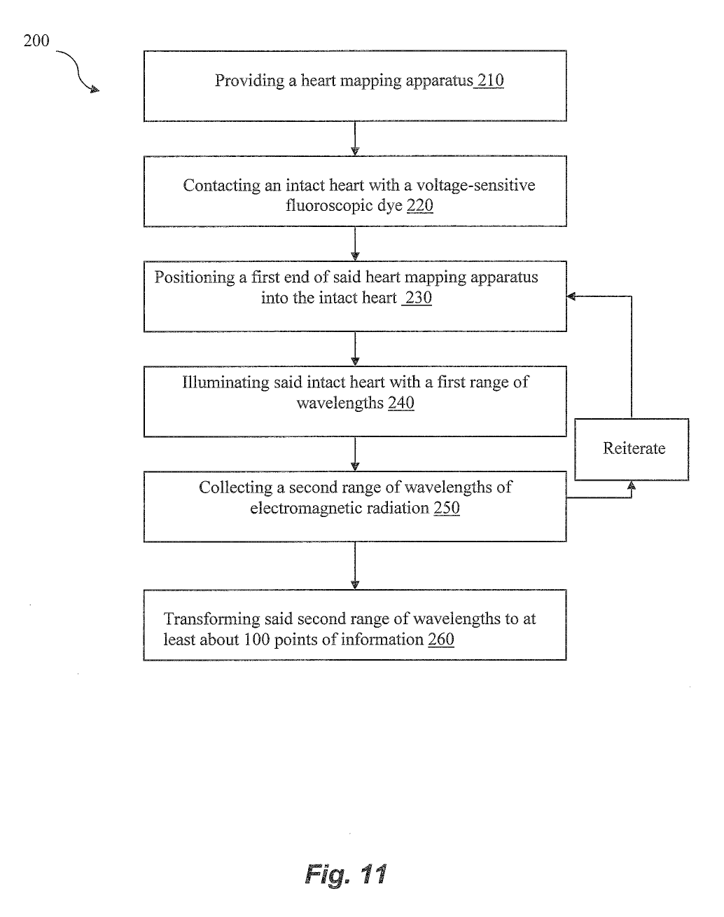

As another example, the step of inserting the posable tubing may be reiterated, as depicted in FIG. 11, in order to, for example, change the location of the heart that is being measured and mapped. For example, while the heart is stained with dye, one or more chambers, including different sides of the heart, may be measured such that re-insertion may be necessary (i.e. there is no cross-over from one side of the heart to the other.)

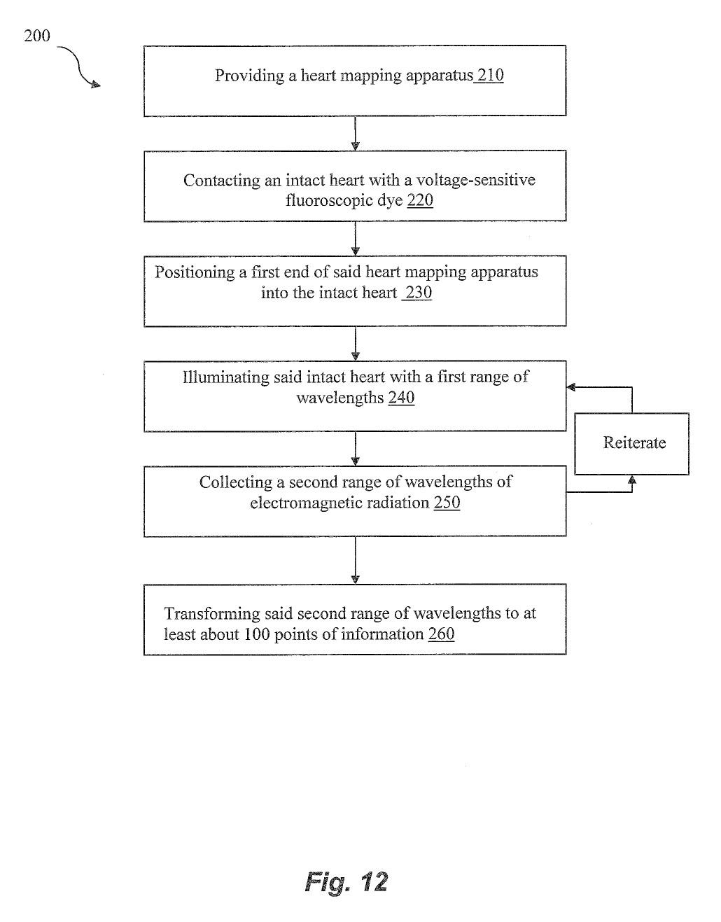

As yet another example, the method 200 may be reiterated from the illuminating step, as shown in FIG. 12. This may allow for a switching of ER sources 110 as a primary illumination with an ER source 110 at, for example, a wavelength of visible light where the ER source is a light bulb followed by a wavelength of infrared light where the ER source is a laser system.

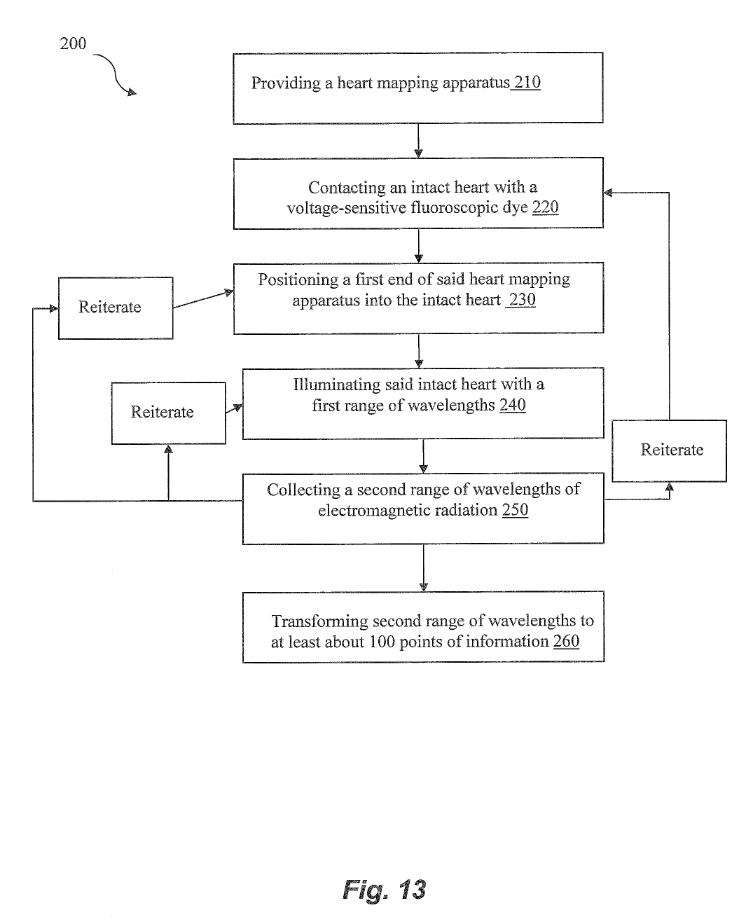

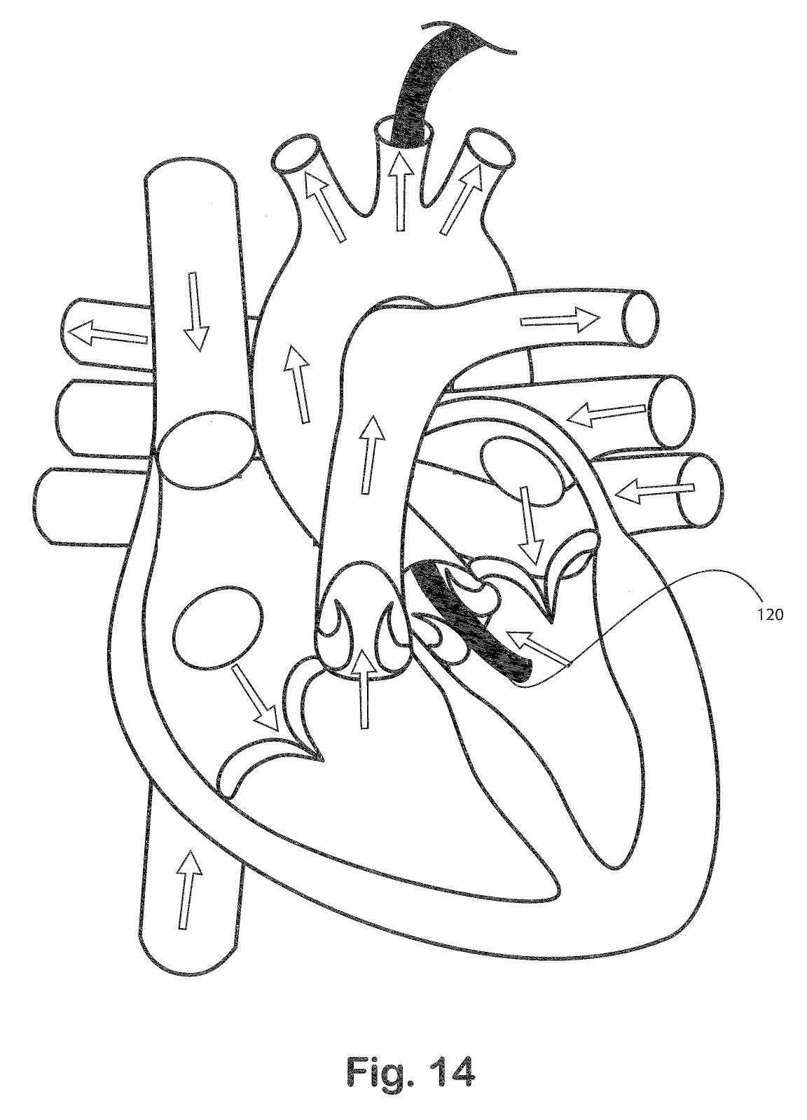

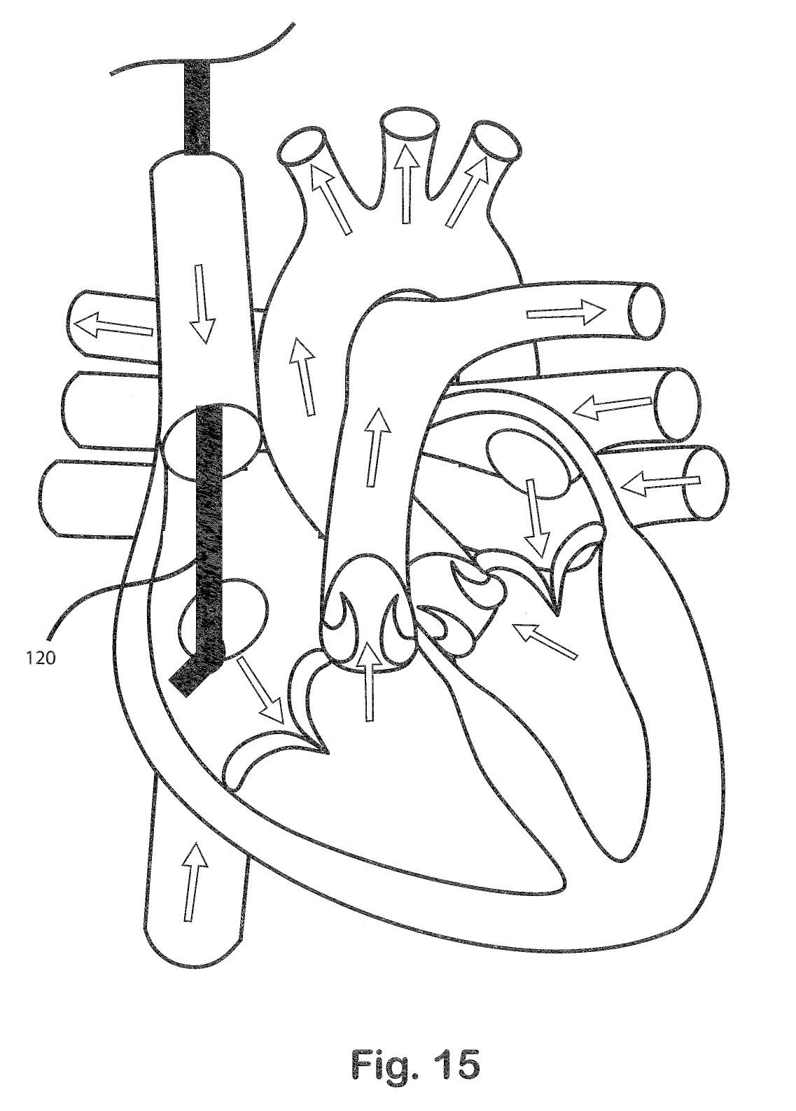

As still yet another example, each of the steps, including the contacting step, inserting/positioning step, illuminating step, and collecting steps may be reiterated as is shown in FIG. 13. This may be done, for example, when testing various variables in a subject heart during one screening session.

The method 200 may provide, in application, greater amount of information and information with a greater detail essential to the accurate screening, analysis, and diagnosis of heart conditions without surgical compromise of the heart itself. This method may be used, for example, as an application in order to screen for, diagnose, or monitor treatment of heart conditions including, cardiac arrhythmias.

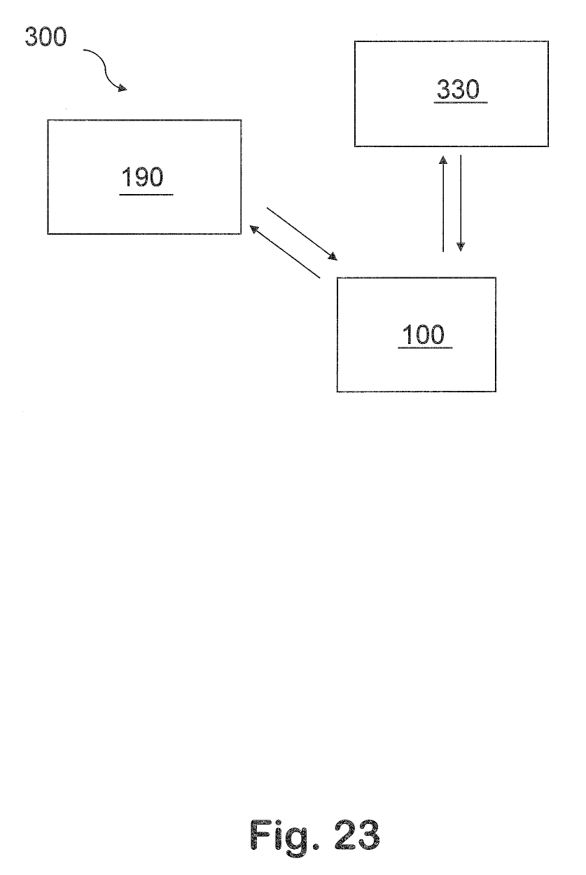

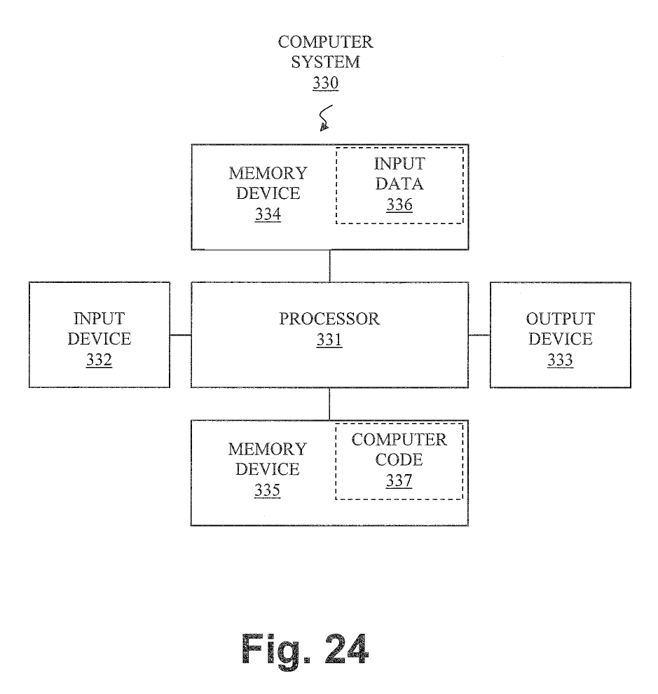

A further embodiment of the present invention also provides a system of high-resolution endocardial mapping 300. The system of endocardial mapping may be shown and depicted in FIG. 23 through FIG. 24, while the computer system of the present invention may be shown in FIG. 24. The system of high-resolution endocardial mapping may comprise, for example: a predetermined amount of voltage-sensitive fluoroscopic dye 190, a heart mapping apparatus 100, and a computer system 330.

The system of high-resolution endocardial mapping 300 may comprise a predetermined amount of voltage-sensitive fluoroscopic dye 190. The voltage-sensitive fluoroscopic dye 190 may be one or more of the dyes disclosed supra. For example, the voltage-sensitive fluoroscopic dye 190 my further comprise a dye which, upon excitation, emits electromagnetic energy at a wavelength of about 750 to about 1800 nm. The predetermined amount may be related to the solubility limit of the dye in the one or more materials, the toxicity level of the dye to cells and in vivo organisms, the concentration of dye needed to get an optimal measurement, the washout time, and similar other factors and considerations. Also, the voltage-sensitive fluoroscopic dye 190 may be configured to absorb into at least a portion of heart tissue 70.

The system of high-resolution endocaridal mapping 300 depicted in FIG. 23 may also comprise a heart mapping apparatus 100. The heart mapping apparatus 100 may include those elements and features disclosed and discussed supra with relation to the heart mapping apparatus. The heart mapping apparatus 100 may be configured to cooperate with said dye to excite said dye such that said dye emits an emission wavelength 60 range higher than the excitation wavelength 50 range, said heart mapping apparatus 100 further configured to transform said emission wavelength range to computer usable data 92.

The system of high-resolution endocardial mapping 300 may further comprise a computer system 330 comprising an algorithm 340, said computer system 330 connected to said heart mapping apparatus 100 and configured to receive said computer usable data 92 and display said data 92 as a simultaneous anatomical feature map and electrical potential map of said portion of said heart tissue.

The computer system may be depicted, for example, in FIG. 24. The computer system 330 comprises a processor 331, an input device 332 coupled to the processor 331, an output device 333 coupled to the processor 331, and memory devices 334 and 335 each coupled to the processor 331.

The input device 332 may be, inter alia, a keyboard, a mouse, a keypad, a touchscreen, a voice recognition device, a sensor, a CCD camera or CMOS camera or array of photodiodes, a network interface card (NIC), a Voice/video over Internet Protocol (VOIP) adapter, a wireless adapter, a telephone adapter, a dedicated circuit adapter, etc.

The output device 333 may be, inter alia, a printer, a plotter, a computer screen, a magnetic tape, a removable hard disk, a floppy disk, a NIC, a VOIP adapter, a wireless adapter, a telephone adapter, a dedicated circuit adapter, an audio and/or visual signal generator, a light emitting diode (LED), etc.

The memory devices 334 and 335 may be, inter alia, a cache, a dynamic random access memory (DRAM), a read-only memory (ROM), a hard disk, a floppy disk, a magnetic tape, an optical storage such as a compact disc (CD) or a digital video disc (DVD), etc. The memory device 335 includes a computer code 337 that is a computer program 340 that comprises computer-executable instructions. The computer code 337 includes, inter alia, an algorithm used for high resolution endocardial mapping according to the present invention. The processor 331 executes the computer code 337. The memory device 334 includes input data 336. The input data 336 includes input required by the computer code 337. The output device 333 displays output from the computer code 337. Either or both memory devices 334 and 335 (or one or more additional memory devices not shown in FIG. 24) may be used as a computer usable medium (or a computer readable medium or a program storage device) having a computer readable program embodied therein and/or having other data stored therein, wherein the computer readable program comprises the computer code 337. Generally, a computer program product (or, alternatively, an article of manufacture) of the computer system 330 may comprise said computer usable medium (or said program storage device).

Any of the components of the present invention can be deployed, managed, serviced, etc. by a service provider that offers to deploy or integrate computing infrastructure with respect to a process for high resolution endocardial mapping of the present invention. Thus, the present invention discloses a process for supporting computer infrastructure, comprising integrating, hosting, maintaining and deploying computer-readable code into a computing system (e.g., computing system 330), wherein the code in combination with the computing system is capable of performing a method for high resolution mapping of a heart or simultaneously mapping the anatomical features and electrical potential of a portion of heart tissue.

In another embodiment, the invention provides a business method that performs the process steps of the invention on a subscription, advertising and/or fee basis. That is, a service provider, such as a Solution Integrator, can offer to create, maintain, support, etc. a process for maintaining a database of heart maps of the present invention. In this case, the service provider can create, maintain, support, etc. a computer infrastructure that performs the process steps and medical diagnostic information (i.e. database) of the invention for one or more customers. In return, the service provider can receive payment from the customer(s) under a subscription and/or fee agreement, and/or the service provider can receive payment from the sale of advertising content to one or more third parties.

While FIG. 24 shows the computer system 330 as a particular configuration of hardware and software, any configuration of hardware and software, as would be known to a person of ordinary skill in the art, may be utilized for the purposes stated supra in conjunction with the particular computer system 330 of FIG. 24. For example, the memory devices 334 and 335 may be portions of a single memory device rather than separate memory devices.

While particular embodiments of the computer system 330 have been described herein for purposes of illustration, many modifications and changes will become apparent to those skilled in the art.

The computer system 330 may further comprise a computer program or algorithm in the form of a database configured to allow a user to save a plurality of heart images, to manipulate said images, and to perform processing functions on the images with said computer system 330.

The computer system 330 of the system of high-resolution endocardial mapping 300 may further comprise an output data, said output data selected from the group consisting of: a three-dimensional image of a portion of the heart; a two-dimensional image of a portion of the heart; a cross-sectional image of a portion of the heart; a numerical table of data corresponding to at least one measurement of heart; a chart of data corresponding to at least one measurement of the heart; a raw data corresponding to at least one measurement of the heart, and combinations thereof.

The system 300 further comprises the display is an instantaneous reading and representation of both at least one anatomical feature of the heart and at least one electrical potential measurement of the portion of heart tissue. Also, the display may be selected from the group consisting of: a video film, a digital photograph, a photograph, a computer generated image, an X-ray, a paper, and combinations thereof.

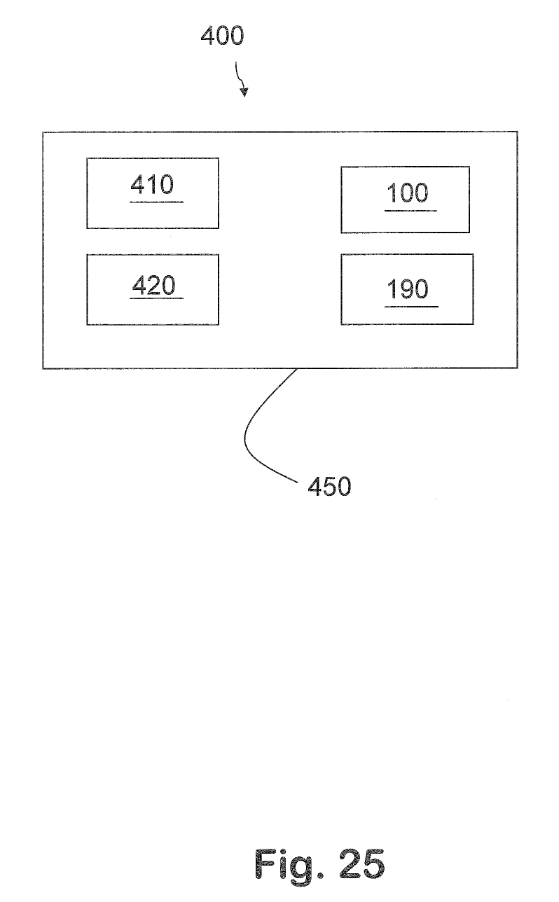

Another aspect of the present invention is the embodiment of a heart mapping kit. The heart mapping kit 400 may be depicted, for example, in FIG. 25. The heart mapping kit 400 may comprise, for example, a predetermined quantity of voltage-sensitive fluorescing dye 190; an administration device 410 for administering to a subject said predetermined quantity of voltage-sensitive fluorescing dye 190; a heart mapping apparatus 100, configured to insert into a circulatory system and enter a heart, further configured to transform an electromagnetic radiation measurement into a data stream; and a computational tool 420, configured to accept said data stream from the heart mapping apparatus and allow a user to analyze, manipulate, and report a result from said data stream. The computational tool may be a computer program, algorithm, or database which aids a user in analyzing, manipulating, and/or reporting from said data stream. The computational tool may be in any computer readable medium, including, for example, CD-ROM, flash drive, floppy disk or hard disk form. Additionally, the kit may comprise a kit casing 440 and/or a relevant information indicator 450 as may be depicted in FIG. 25.

The kit casing 450 may be one integral component or alternatively more than one component that is constructed to configure a single, kit casing 450 in use. The kit casing 450 may be depicted, for example, in FIG. 25. The kit casing 450 may be composed of, for example, but not limited to: plastic, polymer, vinyl, ceramic, glass, fabric, cardboard, metal, wood, woven materials, and combinations thereof. The kit casing 450 may be opaque, translucent, transparent, or a combination thereof. Further, the kit casing 450 may be configured to allow the components of the kit 400 to be completed enclosed and secured therewith. It should be noted that the kit casing 450 may be constructed in any shape and may exist in varying sizes or dimensions.

The kit may further comprise a relevant information indicator 440, as shown in FIG. 26, which may adhere, attach, affix, or associate to at least a portion of the kit 400 or kit casing 450. The relevant information indicator 440 may provide written or pictorial instructions to a user, material safety data sheet information about the voltage-sensitive fluoroscopic dye 190 or solubilizing agents, toxicity information, manufacturer contact information, and/or instructions to aid and/or inform the user in operating the heart mapping apparatus 100, administration device 410, or applications/procedures to use the kit 400.

While many of the benefits of the present invention have been clearly outlined throughout the preceding paragraphs, several of the benefits which have not already been specifically articulated will be herein. The heart mapping apparatus 100 and system of high-resolution heart mapping 300 allow for simultaneous endocardial visualization of the anatomy and the spatiotemporal activation patterns. The signal to noise ratio of the apparatus and its use in the system is comparable to current methods of mapping, with the benefit of surface scanning and multi-level focusing to explore further and deeper areas of initial interest. With such capabilities, the apparatus 100, method 200, system 300, and kit 400 provided each provide valuable tools to map cardiac electrical activity during sinus rhythm and atrial fibrillation in animals, including large animals.

Also, the apparatus 100, system 300, method 200, and kit 400 provide access to areas of the heart for mapping that have previously been unavailable for mapping. That is, the present invention provides access to, for example, small, tight areas around valves and wall corners, and may also, for example provide access to tissue which is not accessible from the epicardium. Not only does the present invention provide for tight and small space mapping, but also the present invention provides maps of much higher resolution levels of the fluorescence-related epicardial electrical activity.

Though previously mentioned, it should also be noted that the imaging approach by the present invention enables anatomy and activation mapping on the endocardium with a spatiotemporal resolution that allows the study of wave propagation in the intact atria. Further, the maps obtained as an element of the present invention are done on an intact heart that is significantly devoid of impaired perfusion and artificial boundaries and amenable for intra-atrial pressure and temperature control (in vivo application). The mapping data acquired excels past the previous mapping approaches and results, and may facilitate the creation of a database to aid with screening, diagnosing, and treating patients who may be affected with one or more heart condition.

Further, the heart mapping apparatus 100, method 200, system 300, and kit 400 may be likewise applied to both of the endocardium and the epicardium, simultaneously mapping the heart. Also, as the heart has complex endocardial anatomy, a high-resolution image or map of the unique anatomy of the heart's surface may provide better spatial markers to thereby provide the present invention with highly efficient retrospective motion correction. The new technology allows the basic biomedical science community to map simultaneously and with high resolution the electrical activity on the internal surfaces of the intact heart. It gives a direct measure of the transmembrane potential of the cardiac cells and allows a more rigorous and relevant study of the dynamics of impulse propagation during arrhythmias. The developed method provides, without the surgical compromise needed today, a more robust and detailed information essential for accurate analysis and diagnosis of cardiac arrhythmias.

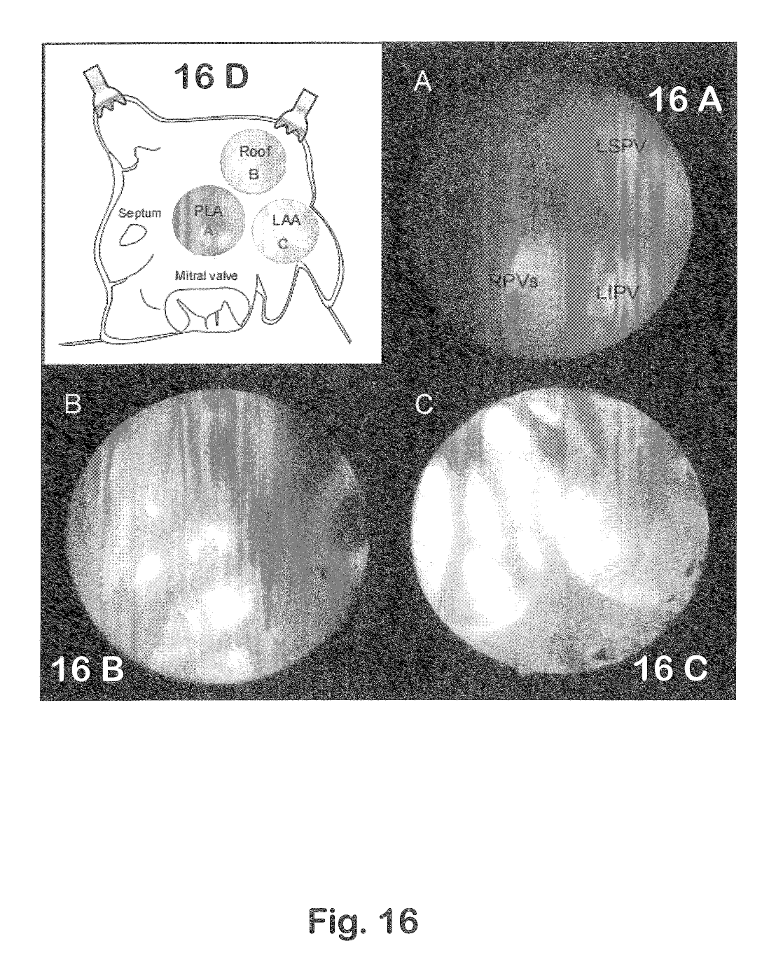

As described and disclosed supra, various experiment related to the present invention were conducted, and the results recorded as is provided by a feature of the present invention. Herein, the pictorial representations and results will be disclosed and discussed, with reference to the FIGs. to which they pertain. The following includes reference to FIG. 16 through 21. Though the present invention is capable of acquiring and displaying information and feedback in color and in live video, the papers provided herewith depict references to still frames and are advanced in grayscale. Where color is referenced, the respective figures are marked up accordingly to illustrate and depict where the color contrast lies.

FIG. 16 through FIG. 21, depict several examples of high-resolution data streams taken at the posterior left atrium (PLA). The various figures of PLA recordings detail the wave propagation patterns during heart conditions including both sinus rhythm and atrial fibrillation. The maps may be acquired with signal-to-noise ratios similar to the ratios of the previously available technology for measuring the heart. However, the heart mapping apparatus 100, method, kit, and system in operation have the ability to visualize highly organized atrial fibrillation sources (also referred to as rotors) at specific locations on the PLA and PLA-pulmonary vein junctions. Further, the maps obtained as an element of the present invention are done on an intact heart that is significantly devoid of impaired perfusion and artificial boundaries and amenable for intra-atrial pressure and temperature control (in vivo application). The mapping data acquired excels past the previous mapping approaches and results, and may facilitate the creation of a database to aid with screening, diagnosing, and treating patients who may be affected with one or more heart condition.

Further, the heart mapping apparatus 100, method 200, system 300, and kit 400 may be likewise applied to both of the endocardium and the epicardium, simultaneously mapping the heart. Also, as the heart has complex endocardial anatomy, a high-resolution image or map of the unique anatomy of the heart's surface may provide better spatial markers to thereby provide the present invention with highly efficient retrospective motion correction. The new technology allows the basic biomedical science community to map simultaneously and with high resolution the electrical activity on the internal surfaces of the intact heart. It gives a direct measure of the transmembrane potential of the cardiac cells and allows a more rigorous and relevant study of the dynamics of impulse propagation during arrhythmias. The developed method provides, without the surgical compromise needed today, a more robust and detailed information essential for accurate analysis and diagnosis of cardiac arrhythmias.

Also, the apparatus 100, system 300, method 200, and kit 400 provide access to areas of the heart for mapping that have previously been unavailable for mapping. That is, the present invention provides access to, for example, small, tight areas around valves and wall corners, and may also, for example provide access to tissue which is not accessible from the epicardium. Not only does the present invention provide for tight and small space mapping, but also the present invention provides maps of much higher resolution levels of the fluorescence-related epicardial electrical activity.

As described and disclosed supra, various experiment related to the present invention were conducted, and the results recorded as is provided by a feature of the present invention. Herein, the pictorial representations and results will be disclosed and discussed, with reference to the FIGs. to which they pertain. The following includes reference to FIG. 16 through 21. Though the present invention is capable of acquiring and displaying information and feedback in color and in live video, the papers provided herewith depict references to still frames and are advanced in grayscale. Where color is referenced, the respective figures are marked up accordingly to illustrate and depict where the color contrast lies.

FIGS. 16A, 16B, and 16C depict successive views of the posterior left atrium and an illustration 16D referencing where the images were taken. The views were taken for the illustrative purpose of establishing the level of resolution that can be acquired by imaging intact atria with the provided heart mapping apparatus and method for high-resolution mapping of a heart. For illustration purposes, the areas that were imaged by the cardio-endoscope in intact atria, are represented after surgical opening and exposure of anatomical features. Three successive views of the PLA in the same heart were obtained by roving the deflectable tip of the endoscope. FIG. 16A: PLA view. FIG. 16B: Roof view. FIG. 16C: LAA view.

FIG. 16A, FIG. 16B and FIG. 16C shows, in grayscale colors, the still-camera registration of the endocardial anatomy as seen through the endoscope in three sample regions of the same heart: The PLA with a view of the PV ostia in FIG. 16A (Panel A), the roof in FIG. 16B (Panel B) and the LAA FIG. 16C (Panel C).

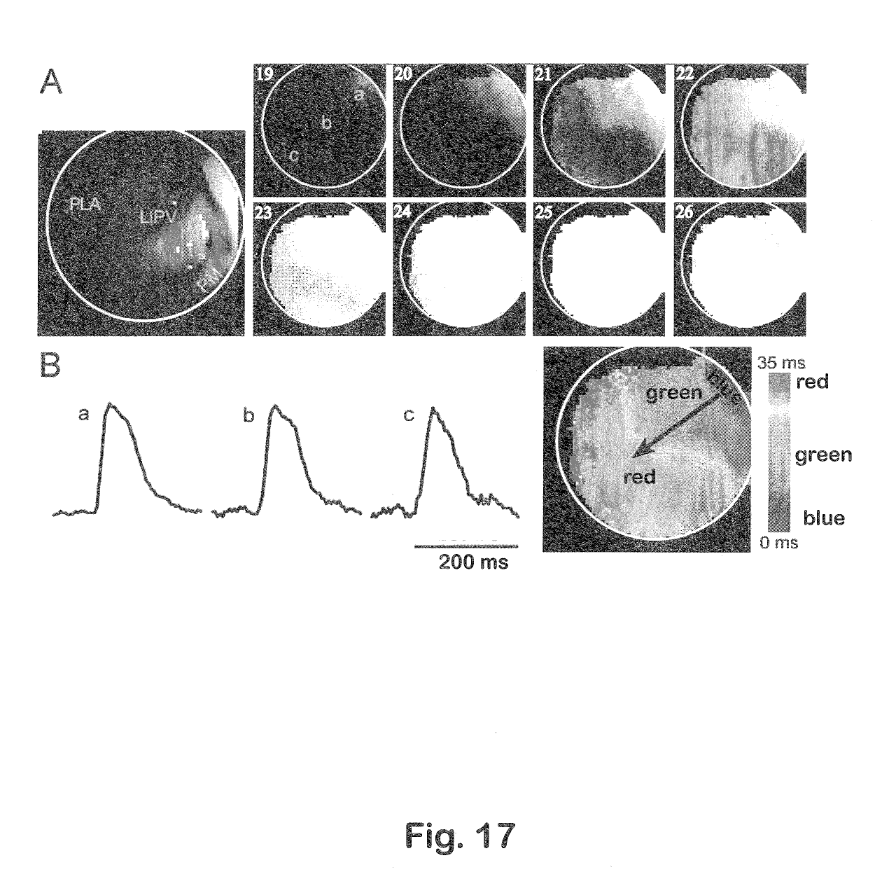

FIG. 17A depicts a fluorescence image of the lower PLA-appendage junction including the LIPV ostium and pectinate muscles (PM) and consecutively obtained frames of an ensemble-averaged movie of sinus impulse propagation. The increased fluorescence of the wavefront is depicted in white while the resting state is in black. Frame numbers are indicated on the upper left corner of each snapshot (300 fr/sec). FIG. 17B depicts single pixel recordings at locations a, b and c (arbitrary units, a.u, of fluorescence) of frame 19 in FIG. 17A and activation map corresponding to the sinus wave propagation (the direction of activation is shown by a black arrow).

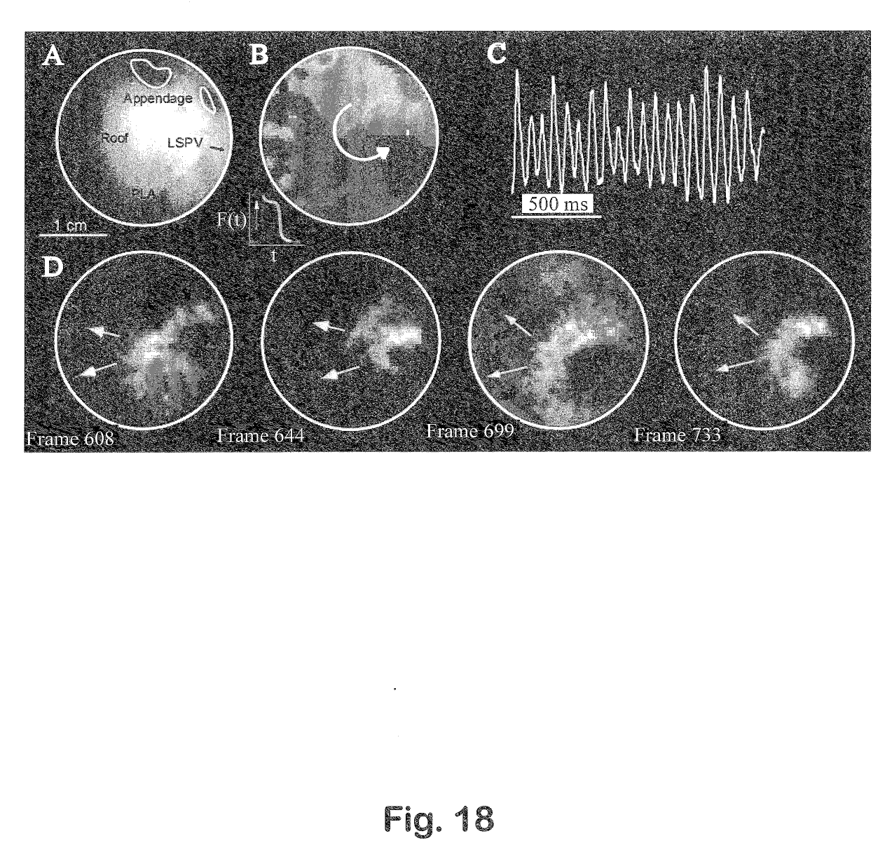

FIG. 18A depicts an endoscopic view (posable tubing 120) of the junction between the roof, the LSPV ridge and the LAA (depicted in FIG. 16B). FIG. 18B depicts a clockwise micro-reentrant activity in a snapshot from a phase movie (phases previously color coded according to the insert appear as contrast in grey-scale). FIG. 18C depicts a single pixel recording in this area exhibited very regular deflections. FIG. 18D depicts this activity transitioned into spatio-temporally organized waves traveling in the septal direction (fluorescent movie illustrated in grayscale contrast, lower panels depicting various frames).

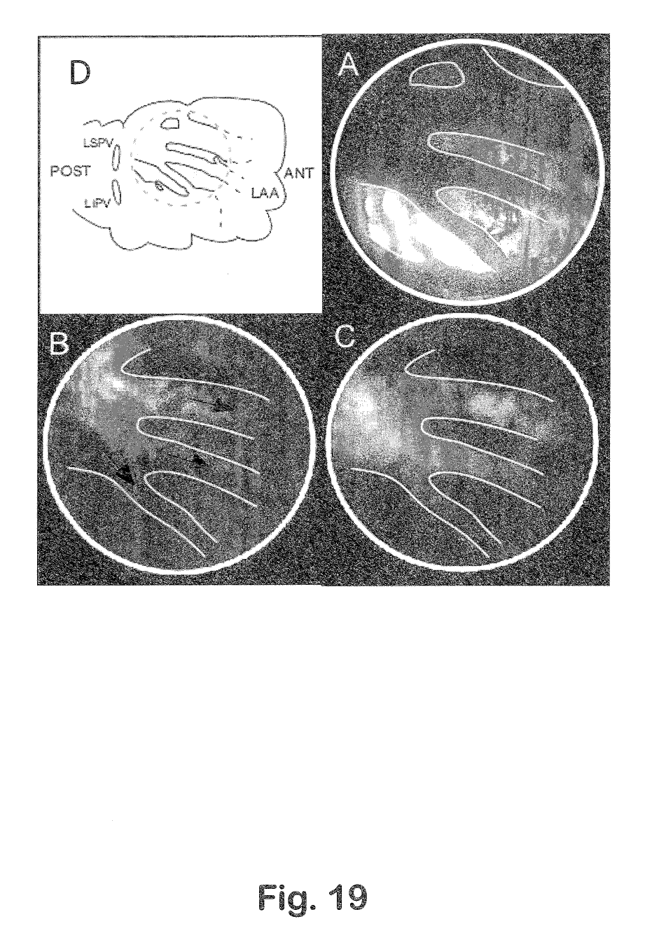

FIG. 19A through FIG. 19C depicts the sinus wave propagation on pectinate muscles. The tip of the endoscope is focused on the circled area, as depicted in the illustration of FIG. 19D, at the junction between the PLA and LAA. FIG. 19B, FIG. 19C depict two consecutive florescence snapshots showing sinus wave propagation of the wavefront through three neighboring pectinate muscles bundles, labeled by white outline. Labeling on FIG. 19D is as follows: ANT, anterior; POST, posterior. The arrows on FIG. 19B depict the direction of propagation, while (as stated) the white lining is to depict the boundaries of muscle bundles.

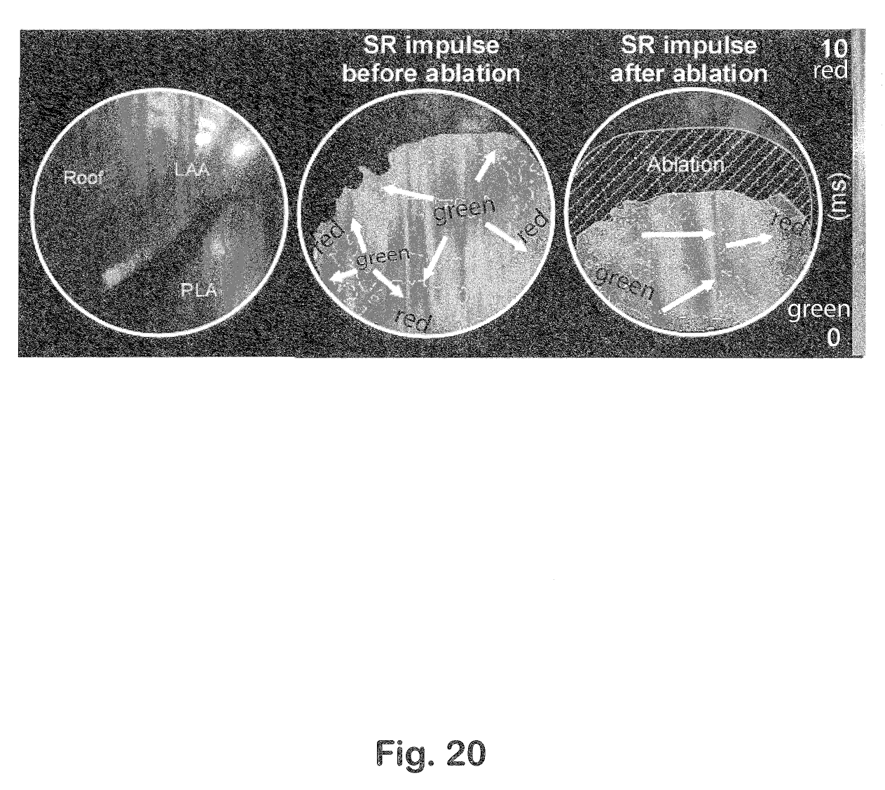

FIG. 20 depicts the visualization of the RF energy delivery effect on sinus rhythm (SR) impulse propagation at the PLA. In the left panel, the ablation catheter is introduced through the working channel 128 of the posable tubing 120 endoscope 121 and seen through its optical channel. The center panel depicts the activation map of a sinus rhythm impulse before ablation. The right panel depicts an activation map of a SR impulse after RF delivery inside the hatched area.

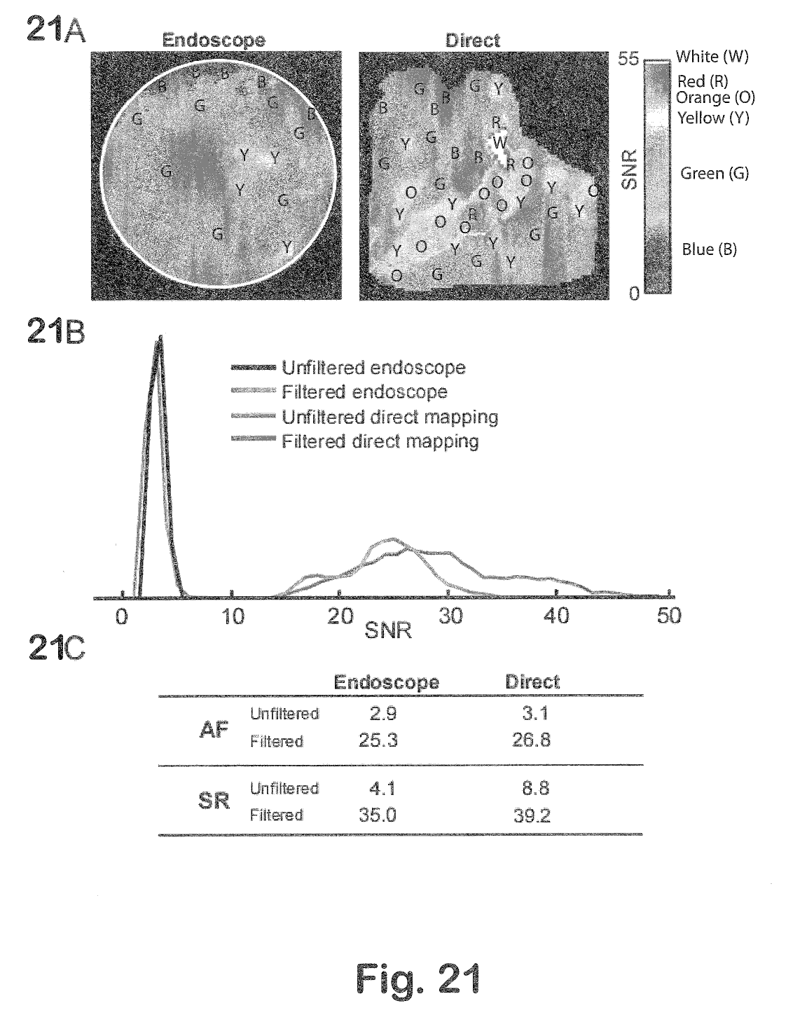

FIG. 21A depicts signal to noise (SNR) maps for filtered atrial fibrillation video recorded from the PLA with the endoscopic mapping device (left panel) and recorded using a conventional optical mapping system from a similar area after having opened the LAA (right panel). The color-coded results are displayed in contrast and labeled accordingly on each panel, left and right. In the endoscopic map, black pixels represent pixels outside the circular field of view; in the direct map, black pixels are pixels outside the PLA and below 10% of maximal amplitude. FIG. 21B depicts signal to noise (SNR) histograms during AF for unfiltered (maps not shown) and filtered data (maps depicted in FIG. 21A; black pixels excluded) obtained for the endoscopic and direct mapping. FIG. 21C provides a table listing the SNR averages calculated from the full width half height FWHH range in the histogram for each condition. SNR averages for AF correspond to data presented in FIG. 21B.

### EXAMPLE

### An Experimental System

The system consists of a direct or side-view endoscope coupled to a 532 nm excitation Laser for illumination, and to a CCD camera for imaging of potentiometric dye fluorescence (DI-4-ANEPPS, 80×80 pixels, 200-800 frames/sec). The cardio-endoscope was aimed successively at diverse posterior left atrial (PLA) locations to obtain high resolution movies of electrical wave propagation, as well as detailed endocardial anatomical features, in the presence and the absence of atrial stretch.

### EXAMPLE

### Stretch-Induced Atrial Fibrillation (AF) Model

Animals were used according to National Institutes of Health guidelines. Young sheep (18-25 kg) were anesthetized with pentobarbital (35 mg/kg intravenously (IV)). Hearts were removed, placed in cold cardioplegic solution, and connected to a Langendorff apparatus. The coronary arteries were continuously perfused at 200 mL/min via a cannula in the aortic root with warm (36 to 38 degrees C.) Tyrode's solution (pH 7.4) equilibrated with 95% O2/5% CO2. A well-characterized model of stretch-related AF was adapted to the sheep heart. After perforation of the interatrial septum, all venous orifices were closed except for the inferior vena cava. Intra-atrial pressure was monitored by a digital pressure sensor connected through a T-cannula to an open-ended tube that was inserted into the inferior vena cava. By changing the height of the open end of the tube, the intra-atrial pressure was controlled. Ventricular fibrillation was induced and intra-atrial pressure was raised above 10 cm H2O. This approach yielded 100% AF inducibility and sustained episodes of AF (>1 hour) without perfusion of acetylcholine. For mapping, boluses of a voltage sensitive fluorescence dye (Di-4-ANEPPS) and, in a subset of experiments 15 mM diacetyl-monoxime to abolish motion artifacts were injected into the perfusate.

### EXAMPLE

### Endoscopic Fluorescence Mapping Set-Up

The heart mapping apparatus has a dual-channel flexible and steerable endoscope (posable tubing). To achieve fluorescence mapping of cardiac impulses the endoscope is coupled to an excitation 532 nm Laser (1-5 W, CW Diode-pumped, Millenia Pro 5sJ, Spectra Physics, Inc.) at the proximal end of the illuminating channel (green arrow) and to a 14 bit CCD camera (SciMeasure, Inc) with a 2.2 mm̂2 chip size. The camera is C-coupled with a 12 mm, 1:1.4 maximal N.A. and ⅔″ diagonal field focusing lens to a 645±50 nm band-pass filter and to the eyepiece of the imaging channel (red arrow). The following endoscopes were chosen for mapping the different regions of the LA: (i) a sigmoidoscope (Pentax, Inc., FS-34P2) of 11.5 mm diameter, 120 degrees field of view and 63 cm working length. This endoscope features a deflectable direct view tip (FIG. 5) with angulations of 180°/180° (up/down) and 160 degrees/160 degrees (right/left) or (ii) a therapeutic duodenoscope (Olympus, Inc. JF1T) of 11.0 mm diameter, 80 degrees field of view and 103 cm working length. This endoscope features a deflectable side view tip (FIG. 6) with angulations of 130 degrees/130 degrees up/down and 90 degrees/90 degrees right/left. Endoscopes showed transmittance of about 13% and 11%, respectively, as assessed by a 532 nm laser input in the range of 0.2-5W with a digital power meter (FieldMaster-GS, Coherent, Inc.).

### EXAMPLE

### Mapping Protocols

A small cut was made in the left ventricle, carefully avoiding any visible coronary branches, to introduce the endoscope into the left atrium (LA) through the mitral valve. A digital camera (Kodak Professional, DCS 300, Nikor 50 mm f 1.4 D) was connected to the recording port of the endoscope to adjust the endoscope tip and provide clear images of the mapped region inside the intact LA. For enhanced detection of the anatomical details, external ambient illumination was used in addition to the internal white illumination that replaced the laser light source 111 as needed.

FIG. 16 depicts in realistic colors the still-camera registration of the endocardial anatomy as seen through the endoscope in three sample regions of the same heart: The posterior left atrium (PLA) with a view of the PV ostia (Panel A), the roof (Panel B) and the LAA (Panel C). Five-sec movies (di-4-ANEPPS, 80×80 pixels, 200-800 frames/sec) were recorded during Sinus rhythm (SR), after the endoscope was aligned to visualize the PLA and other left atrial locations. Then, the intra-atrial pressure was set above 10 cm H₂O and sustained AF was readily induced by burst pacing (10 Hz, 10 sec, 5 ms pulse duration, twice threshold).

Thereafter the endoscope was steered to record video stream at a time of 5-sec movies from the roof and the LAA (though longer video may be recorded). In most cases, the Kodak camera was subsequently connected to the eyepiece of the endoscope and color pictures were acquired using large opening times (⅓ to 1/30 sec). It was also possible to visualize the anatomy based upon the background fluorescence image created from the temporal average of the movies. For visualization of the electrical activation, movies were processed with background subtraction, 5-point spatiotemporal averaging, high-pass filtering, as previously described, and additional sequential frames subtraction for the AF movies.

To correct for motion artifacts during AF and SR, a retrospective de-morphing algorithm was applied based on a template-matching technique. The movies obtained showed the activity at the PLA during SR. A comparison of the corrected and non-corrected movies demonstrates the efficiency of the removal of most of the motion artifacts by the template-matching technique. It should be noted that the presence of various endocardial anatomical structures makes the template-matching algorithm for the endoscopic mapping more effective than for the epicardial mapping.

### EXAMPLE

### Signal-to-Noise Ratio Evaluation

The endoscopic mapping system was compared with the direct mapping system by evaluating their respective signal-to-noise ratios (SNRs) during SR and AF. The direct mapping system consisted of the same camera and light source used for the endoscope but arranged in a conventional epifluorescence setting to image the PLA through a minimal LAA incision. In both approaches, signal levels for the respective SR and AF movies were determined as the peak-to-peak amplitude minus twice the noise level. On the other hand, noise levels were calculated as the standard deviation of the peak-to-peak amplitude during quiescent episodes of background subtracted movies for SR, and of background and sequentially subtracted movies for AF. Pixel-by-pixel SNRs were combined in maps generated for both unfiltered and filtered background subtracted data. The SNR of the direct system was determined for movies during both SR and cholinergic AF (0.5 μM ACh) and analyzed as described above. SNR histograms for the pixels in the maps were generated and average SNR values were calculated for the full width half height (FWHH) range.

Experimental Results:

Representative cardio-endoscopic images of LA activation during SR are presented in FIG. 17A and FIG. 17B. The leftmost image in panel of FIG. 17A shows a snapshot of the background fluorescence with the anatomical details of the posterior-lateral LA including the left inferior pulmonary vein ostium (LIPV), pectinate muscle (PM) and free wall and PLA junction (J). This field of view is located between those shown as FIG. 16A and FIG. 16C, and depicted in the illustration may of FIG. 16D in FIG. 16. However, unlike FIG. 16A-D, the anatomical picture in FIG. 17A was obtained by the integral CCD camera. Frames 19 to 26 in FIG. 17A are the average sinus activations taken every 3.33 ms from a fluorescence movie after background subtraction. The grey scale indicates membrane potential level; resting tissue appears in black and excited tissue in white. Labels a-c on frame 19 indicate the locations of the single pixel recordings shown in FIG. 17B. The excellent signal-to-noise ratio of such traces clearly allows detection of the membrane potential changes during SR. The SR color activation map, constructed by measuring the time at which the action potential upstroke reaches 50% amplitude at each pixel location is shown on the right side of FIG. 17B. It shows the impulse traveling from the top right to the lower left edge of the field of view, in a general direction from the roof of the appendage towards the lower septal part of the PLA. This septal-bound propagation direction in the PLA is consistent with previous description of SR activation patterns in humans using a non-contact mapping system and supports the relevance of the mapping approach of the present invention.

FIG. 18A through 18D shows an example of left atrial impulse propagation during stretch-related AF. The tip of the endoscope was directed toward the PLA roof at its junction with the LAA (depicted as FIG. 18A), in the vicinity of the ridge of the left superior PV (LSPV, similar to location B in FIG. 16B but seen through the CCD in grayscale). Spatiotemporally organized waves were observed during most of the five-sec movie. A phase movie (action potential phases were color coded according to the inset) snapshot depicts a micro-reentrant wave rotating counter-clockwise at a frequency of 11.7 Hz (FIG. 18 B, labeled as B). As shown by the single pixel recording in panel C of FIG. 18C, this rotor exhibited regular deflections. The rotational activity transformed into spatiotemporally organized waves traveling in the septal direction. FIG. 18D shows four sequential patterns of activation with the same direction and averaged interbeat cycle length of 74 ms. In addition to being able to image spatiotemporally repetitive AF waves originating from the PLA-LAA junction, it is also possible to record impulses emanating from this location and traveling into the pectinate muscles.

FIGS. 19B and 19C provide an example of propagation of AF waves through LAA pectinate muscle bundles, at their connection with the PLA (diagram provided in FIG. 19D and FIG. 19A) is presented. Most of the waves propagated in the direction PV-to-LAA (posterior to anterior) as with other AF model and epicardial mapping. Also, some of the waves propagated in the opposite direction (LAA-to-PV) or else traveled vertically from top to bottom of the field-of-view. Interestingly, some of the wavefronts were substantially delayed as they propagated through the lowest pectinate bundle in comparison with the two higher bundles in view. Overall, the endocardial data shown in FIG. 18A-D and FIG. 19A-D establishes that in addition to being a source for LAA-bound activity, the PLA-LAA roof junction also generates oppositely PLA-bound activity, as shown endocardially in FIG. 19A-FIG. 19D.

### EXAMPLE

### Radio-Frequency (RF) Ablation at the PLA

Direct visualization of the anatomical structure and the electrical activity through the same endoscope should improve guidance of ablation procedures. The left panel of FIG. 20 shows the tip of an ablative catheter (4F, 105 cm, Biosense Webster, Inc.) introduced through the working channel 128 and viewed through the optical channel of the endoscope. The center panel shows an isochrone map. Before the ablation catheter was inserted into the endoscope, activation of the PLA during SR consisted of an extended breakthrough that spanned from the center of the PLA, close to the LSPV ostium, to the septum. Thereafter, the catheter was introduced into the endoscope and following electrode-tissue contact verification, 30-35 W of RF energy was delivered for 30-60 sec while the tip was dragged on the upper part of the PLA (right panel; hatched area). As shown by the right panel, application of RF, dramatically changed the SR impulse propagation pattern. The activity appeared at the RIPV area and then traveled in the septum-to-LAA direction.

Signal to Noise (SNR) Evaluation

FIGS. 21A through 21C presents SNR maps constructed after pixel-by-pixel analysis during AF after filtration of the signal for the endoscopic system (left) and the direct mapping system (right) in two different hearts. In general, the SNR of the cardio-endoscopic recording is more homogeneous than the SNR of the direct mapping recording, except for the upper area that corresponds to the PLA-roof transition. This difference in distribution of SNR may be attributed to the different orientation of the excitation light beam relative to the optical axis of the objectives in the two systems. FIG. 21B shows the SNR histogram for the two maps shown in FIG. 21A and also from additional SNR maps obtained for unfiltered, raw movies. Comparison between the histograms of the two systems shows that they generally overlap. The table in FIG. 21C demonstrated that during AF the average unfiltered SNRs are 2.9 and 3.1 and 25.3 and 26.8 after filtration, respectively for the endoscopic and direct systems. Also shown in this table are data obtained during SR (unfiltered average SNRs were 4.1 vs. 8.8 for endoscopic and direct system, respectively, and 35 vs. 39.2 for filtered data). Overall, the SNR achieved for the cardio-endoscopic system is only slightly lower than the direct mapping system. In fact, during AF the difference is negligible. This demonstrates that the cardio-endoscopic device permits adequate conditions for mapping.

Endoscopic Mapping and Stretch-Related AF Model

While movies were obtained from the LA during AF and SR in conventional Langendorff-perfused sheep hearts in the unstretched atria, the application of stretch in this model yielded major positive outcomes. Increasing the left atrial pressure above 10 cm H₂O rendered AF readily inducible without the utilization of acetylcholine. Also, the resultant anatomical expansion facilitated the process of repositioning the endoscope to visualize various areas of the LA through maneuvering the endoscopic deflection and orientation. Further, the increased intra-atrial pressure forced most air bubbles out of the LA, hence reducing optical distortion. Thus, the heart mapping apparatus 100 is ideally suited to investigate, inter alia, stretch-related arrhythmias and other heart conditions.

Various modifications and variations of the described apparatus, kit, method, and system of the invention will be apparent to those skilled in the art without departing from the scope and spirit of the invention. Although the invention has been described in connection with specific embodiments outlined above, it should be understood that the invention should not be unduly limited to such specific embodiments. Various changes may be made without departing from the spirit and scope of the invention as defined in the following claims.

We claim:

1. A heart mapping apparatus, comprising:
an electromagnetic radiation source capable of exciting a fluoroscopic dye, said dye configured to emit at least an excitation wavelength of electromagnetic radiation;
a posable tubing, configured to associate with said electromagnetic radiation source, said posable tubing including:
a delivery channel operable to deliver said excitation wavelength of electromagnetic radiation from said electromagnetic radiation source to a portion of heart tissue dyed with said fluoroscopic dye and
an acquisition channel operable to acquire electromagnetic radiation having at least an emission wavelength defined by electromagnetic radiation, said emission wavelength emitted from said portion of heart tissue dyed with said fluoroscopic dye;
an acquisition member, configured to receive electromagnetic radiation at the emission wavelength and transform the acquisition wavelength into a data stream having at least 100 points of information.

2. The apparatus of claim 1, wherein the apparatus further comprises a filter, said filter configured to associate with said acquisition member.

3. The apparatus of claim 1, wherein the electromagnetic radiation source is a laser.

4. The apparatus of claim 1, wherein the posable tubing is a catheter having at least two channels.

5. The apparatus of claim 1, wherein the delivery channel and the acquisition channel further comprise a delivery fiber optic bundle cable and an acquisition fiber optic bundle cable.

6. The apparatus of claim 1, wherein the acquisition member further comprises one selected from the group consisting of: a charge coupled device (CCD) camera, a complementary metal-oxide-semiconductor (CMOS) camera, a photodiodes array, and combinations thereof.

7. A method of high-resolution mapping of a heart, comprising:
providing a heart mapping apparatus;
contacting an intact heart with a voltage-sensitive fluoroscopic dye to generate at least a portion of dyed heart tissue;
positioning a first end of said heart mapping apparatus into the intact heart;
illuminating said intact heart with a first range of wavelengths of electromagnetic radiation from said first end of said heart mapping apparatus;
collecting a second range of wavelengths of electromagnetic radiation from said portion of dyed heart tissue with said first end of said heart mapping apparatus; and
transforming said second range of wavelengths of electromagnetic radiation to at least about 100 points of information, where the points of information are a map at least one anatomical features and one electrical potential.

8. The method of claim 7, wherein the inserting step further comprises in vivo inserting into an entry point of a subject and following the circulatory system to the heart, said entry point selected from the group consisting of: a femoral artery, a carotid artery, a jugular vein, and a brachial artery.

9. The method of claim 7, wherein the collecting step further comprises collecting and transmitting by fiber optic channel the second range of wavelengths to a second end of the heart mapping apparatus.

10. The method of claim 7, wherein the transforming step further comprises transforming by a CCD camera the second range of wavelengths into a usable data.

11. The method of claim 7, wherein the illuminating step further comprises illuminating the intact heart with said first range of wavelengths is from about 500 to about 1800 nm.

12. The method of claim 7, wherein the collecting step further comprises collecting electromagnetic radiation of wavelengths from at least about 600 to about 1800 nm.

13. A system of high-resolution endocardial mapping, comprising:
a predetermined amount of voltage-sensitive fluoroscopic dye, configured to absorb into at least a portion of heart tissue;
a heart mapping apparatus, configured to cooperate with said dye to excite said dye such that said dye emits an emission wavelength range higher than the excitation wavelength range, said heart mapping apparatus further configured to transform said emission wavelength to computer usable data; and
a computer system including an algorithm, said computer system connected to said heart mapping apparatus and configured to receive said computer usable data and display said data as a simultaneous anatomical feature map and electrical potential map of said portion of said heart tissue.

14. The system of claim 13, wherein the computer system further comprises a database configured to allow a user to save a plurality of heart images, to manipulate said images, and to perform processing functions on the images with said computer system.

15. The system of claim 13, wherein the computer system further comprises an output data, said output data selected from the group consisting of: a three-dimensional image of a portion of the heart; a two-dimensional image of a portion of the heart; a cross-sectional image of a portion of the heart; a numerical table of data corresponding to at least one measurement of heart; a chart of data corresponding to at least one measurement of the heart; a raw data corresponding to at least one measurement of the heart, and combinations thereof.

16. The system of claim 13, wherein the voltage-sensitive fluoroscopic dye further comprises a dye which, upon excitation, emits electromagnetic energy at a wavelength of about 600 to about 1800 nm.

17. A heart mapping kit, comprising:
a predetermined quantity of voltage-sensitive fluorescing dye;
an administration device for administering to a subject said predetermined quantity of voltage-sensitive fluorescing dye;
a heart mapping apparatus, configured to insert into a circulatory system and enter a heart, further configured to transform an electromagnetic radiation measurement into a data stream; and
a computational tool, configured to accept said data stream from the heart mapping apparatus and allow a user to analyze, manipulate, and report a result from said data stream.

18. The kit of claim 17 further wherein the voltage-sensitive fluoroscopic dye is a styryl dye.

19. The kit of claim 17 wherein the computational tool further comprises a computer program on a computer readable medium.

20. The kit of claim 17 further comprising a kit casing.
